(19) United States
(12) Patent Application Publication
(10) Pub. No.: US 2026/0325627 A1
(43) Pub. Date: Oct. 22, 2026

(54) IMMUNOLOGICALLY INACTIVE POLY (ETHYLENE GLYCOL) POLYMER DERIVATIVES AND BIOCONJUGATES THEREOF FOR USE AS ALTERNATIVE IN PEGYLATED THERAPEUTICS, PROCESSES FOR THEIR PREPARATION AND THEIR USE

(71) Applicant: JOHANNES GUTENBERG-UNIVERSITÄT MAINZ, Mainz, DE (US)

(72) Inventors: Philip DREIER, Mainz (DE); Rebecca MATTHES, Mainz (DE); Holger FREY, Mainz (DE); Fabian FUSS, Mainz (DE)

(73) Assignee: JOHANNES GUTENBERG-UNIVERSITÄT MAINZ, Mainz, DE (US)

(21) Appl. No.: 19/481,828

(22) PCT Filed: Jun. 12, 2024
(86) PCT No.: PCT/EP2024/066257

§ 371 Date: Nov. 5, 2025

(30) Foreign Application Priority Data
Jun. 14, 2023 (EP) 23179196.3

(51) Int. Cl. A61K 39/385; A61K 39/215; A61K 47/60; A61K 47/69; C08G 63/06; C08G 65/34; C08G 73/02

(52) U.S. Cl. CPC A61K 39/385; A61K 39/215; A61K 47/60; A61K 47/6929; C08G 63/06; C08G 65/34; C08G 73/024

(57) ABSTRACT

Immunological inactive polyether polymers represented by the following formula [I]:

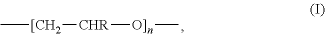

wherein at least 3% of the residues R include ethylene oxide side chains, oligo(ethylene oxide) side chains and/or glycerol ether side chains and at least 3% of the residues R are hydrogen and R may also be a C1 to C3-alkyloxymethyl side chain. Further, a process for the preparation of the polymers. Finally, the use of the polymers for the preparation of conjugates, especially bioconjugates and the conjugates themselves as alternative for PEGylated therapeutics. The conjugates may include bioactive compounds and/or lipids. They are especially useful for the preparation of vaccines, lipid particles, especially lipid nanoparticles and may be used for the preparation of vaccines against COVID-19.

### INTRODUCTION

The present invention concerns immunological inactive polyether polymers represented by the following formula [I]:

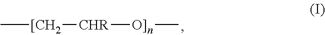

wherein at least 3% of the residues R comprise ethylene oxide side chains, oligo(ethylene oxide) side chains and/or glycerol ether side chains and at least 3% of the residues R are hydrogen and R may also be a C1 to C3-alkyloxymethyl side chain. The invention further concerns a process for the preparation of the polymers. Finally, the invention concerns the use of the polymers for the preparation of conjugates, especially bioconjugates and the conjugates themselves as alternative for PEGylated therapeutics. The conjugates may comprise bioactive compounds and/or lipids. They are especially useful for the preparation of vaccines, lipid particles, especially lipid nanoparticles and may be used for the preparation of vaccines against COVID-19.

### BACKGROUND OF THE INVENTION

The aliphatic polyether poly(ethylene glycol) (PEG) is the most widely employed polymer in pharmaceutics, medicine and drug delivery. PEGylation, i.e. the attachment of PEG to peptide drugs, was introduced in the 1970ies and has since then led to numerous highly efficient drugs. PEGylation is a form of conjugation, i.e. the attachment of a polymer to a molecule by a covalent bond. The main effect of the attachment of PEG chains is the so-called “stealth effect”, which prevents close approach of and recognition by the immune system and subsequent binding and removal by the reticuloendothelial system (RES) in the liver. More simply, PEG attachment also prevents opsonization by proteins, when attached to the surfaces of nanocarriers for drug delivery. The inhibition of protein adsorption by PEG is further emphasized by fulfillment of the rules for protein-repellent surfaces derived by Whitesides (Yarovsky et al., “Quantitative design rules for protein-resistant surface coatings using machine learning”, Scientific Rep. 2019, 9, 265). PEGylation is also used for “stealth liposomes” with long circulation times, low molecular weight drugs as well as for lipid nanoparticles. Such structures are employed for the transport and delivery of mRNA. Here the respective PEG-based lipids play a key role for the vaccination against the COVID-19 pandemic, enabling transport of mRNA to cells, avoiding undesired degradation processes. The recently approved mRNA vaccines from Moderna and Pfizer-BioNTech require PEGylated lipids to ensure the stability of the mRNA in the lipid-based nanoparticle. PEGylation also prevents coalescence of liposomes and similar structures in aqueous systems. Commonly, molecular weights in the range of 2 to 40 kg/mol are preferred for the PEGylation of drugs or nanocarriers. To sum up, PEG has become a standard for water-soluble medical polymers for a vast variety of medical and pharmaceutical uses, particularly in the therapy of cancer, chronic diseases and recently also for RNA delivery vehicles. The success of PEG may have had several reasons. Among them are commercial availability in sufficient quality and in a wide range of molar masses from the anionic ring-opening polymerization of ethylene oxide as well as its extremely low toxicity and high biocompatibility. Further, PEG can be prepared in narrow molecular weight distributions.

However, an increasing number of studies summarize concerns related to the presence of so-called anti-PEG antibodies (APAs) that are present in a large and constantly growing part of the population. Such antibodies can lead to accelerated clearance of PEGylated drugs from the blood stream, i.e. loss of the stealth effect that is crucial for therapeutic success, allergic reactions and in extreme and rare cases even potentially fatal anaphylactic shock. This undesired effect is observed both for PEGylated peptide drugs, PEGylated liposomes and other PEGylated nanocarriers as well as PEGylated small molecule drugs. Anti-PEG antibodies are also problematic with respect to PEGylated lipids for RNA-transporting lipid nanoparticles, e.g. in the context of COVID-19 vaccines. PEG-antibodies are often mentioned in the context of potential PEG alternatives as a motivation to look beyond PEG for alternatives. As an estimate, approximately 10% of the population experience allergic reactions when treated with PEGylated formulations. Only recently, the nature of the binding between PEG and anti-PEG antibodies has been studied by Lai et al (Lai et al, “Structure of an anti-PEG antibody reveals an open ring that captures highly flexible PEG polymers.” Commun Chem 2020, 3, 124). They concluded that binding of PEG is due to an open ring structure of PEG captured by the APAs. By counting the number of monomer repeats of the PEG polymer interacting with the interior and exterior paratope of the Fab, they found the size of the PEG antigen epitope to be ~700 g/mol, equivalent to 16 monomer subunits. Thus, at least 16 ethylene glycol units are required for binding by the APA. Other works suggest that also shorter regular chain segments of PEG as well as the methoxy end group of mPEG (α-methoxy poly(ethylene glycol)) structure play a key role for the recognition. U.S. Pat. No. 8,129,330 B2 therefore discloses methods for the preparation of PEG conjugates that are based on end group modification of the PEG chains. Variation of the end group of PEG is disclosed, aiming at minimizing interaction of for example methoxy end groups with anti-PEG antibodies. The authors disclose conjugates, wherein a hydroxyl group is present on all of the distal polyalkylene glycol termini in said pure conjugate, and wherein said conjugate exhibits reduced antigenicity compared to mPEG-protein conjugates. It has been reported that antibodies elicited by PEG-OH have similar affinity to both mPEG and PEG-OH, while antibodies induced by mPEG recognize mPEG more effectively than PEG-OH (Sherman et al, “Role of the methoxy group in immune responses to mPEG-protein conjugates.” Bioconjug Chem 2012, 23(3), 485-499; Sherman et al, “Selectivity of binding of PEGs and PEG-like oligomers to anti-PEG antibodies induced by methoxyPEG-proteins.” Mol Immunol 2014, 57(2), 236-246.). These results have been obtained by using competitive ELISA and imply that the anti-PEG antibodies elicited by PEG-OH-proteins are directed against the backbone of the PEG (backbone-specific), while antibodies induced by mPEG-protein conjugates are methoxy group specific. It can be concluded that regular segments of at least 4-5 to 16 regularly arranged ethylene glycol units are required to achieve recognition and immunogenic reaction by anti-PEG antibodies. Recent clinical works demonstrated that the presence of antibodies against PEG permits to predict Pegasparagase allergic reactions and failure of rechallenge, emphasizing the clinical importance of anti-PEG antibodies for the success of the treatment of leukemia in this case (Liu et al, “Antibodies Predict Pegasparagase Allergic Reactions and Failure of Rechallenge”, J. Clin. Oncol. 2019, 37, 2051).

Four strategies for the modification of the PEG structure have been developed to counteract undesired APA interaction of PEGylated therapeutics. Replacing the methoxy end group of mPEG by a hydroxyl group led to a strong decrease in the affinity for anti-PEG antibodies. However, it is obvious that for large scale commercial applications the presence of a hydroxyl group at the terminus would require protected functional initiators, which complicates existing synthetic protocols. A second recent strategy is disclosed in US 2019/0015520 A1 and relies on PEG-bottlebrush structures, i.e. small PEG structures appended to a poly(hydroxyethyl methacrylate) backbone. The inventors state that this type of PEG-architecture minimizes anti-PEG antigenicity, while preserving the desired stealth properties for surface coating. However, the resulting PEG-grafted copolymers exhibit rather broad, multimodal molecular weight distributions, which represent a major obstacle for approval for medical application and PEGylation analogous bioconjugation with peptides or lipids. Narrow distributions (Mw/Mn<1.15) were exclusively feasible by performing a cost- and time-consuming preparative size-exclusion chromatography (SEC).

Another strategy is to exchange PEG with the linear polyether polyglycerol to increase the blood circulation time of liposomes and to avoid accelerated blood clearance (see Abu Lila et al, “Use of polyglycerol (PG), instead of polyethylene glycol (PEG), prevents induction of the accelerated blood clearance phenomenon against long-circulating liposomes upon repeated administration”, International Journal of Pharmaceutics, 456 (2013) 235-242). However, the utilization of polyglycerol also results in the introduction of multiple hydroxyl functionalities to the liposome, which is in contrast to the aforementioned rules of Whitesides resulting in complex unspecific recognitions of peptides in the blood stream. Therefore, a targeted use of the conjugated drug or liposomes is not possible with polyglycerol.

A fourth strategy has been presented by the inventors of the present patent application, in which C1 to C3 alkoxymethyl side chains are appended to the backbone of the polymer (see WO 2022/238532 A1 and European patent application No. EP 22 203 748; not yet published). While this is an effective strategy, it has some limitations. Poly(ethylene oxides) with methoxymethyl side chains, e.g.

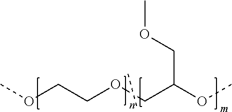

have a hydrophilicity that is practically identical to polyethylene glycol, but the side chains have limited spatial requirements. In poly(ethylene oxides) with C2 and C3 alkoxymethyl side chains, e.g.

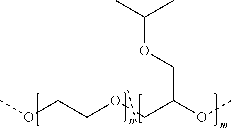

the side chains are more sterically demanding, but the polymers are less hydrophilic. Hydrophilicity is necessary to provide solubility and stealth effect to the polymers. A high steric footprint of the side chains is necessary to prevent crystallization and immunogenic reactions. Increased spatial requirements of the said PEG copolymers impede or disable interaction with anti-PEG antibodies according to the specific “lock and key principle”. Equally important, the use of a PEGylated pharmaceutical agent has shown that the immunogenic system is still not very well understood. It seems that almost any chemical structure may trigger an immunological reaction. There is a need for a variety of tools for the manufacturing PEGylated drugs to choose from. Availability of a variety of polymers suitable for the PEGylation of drugs also provides the opportunity to adapt the polymer to the structure of the physiologically active agent and may thus allow easier production, better delivery and/or improved effect of the drug.

Since PEGylated therapeutics are often used for chronic or in many cases otherwise fatal diseases, like in cancer therapy, in many cases repeated doses have to be administered over prolonged periods of time. PEG is a non-biodegradable polymer and has to be eliminated from the body through the kidneys. In studies on the renal filtration of PEGylated drugs, an undesirable accumulation of PEG in the kidneys and the associated pathological changes in the renal system could be demonstrated (see e.g. Bendele, A.; Seely, J.; Richey, C.; Sennello, G.; Shopp, G. Short Communication: Renal Tubular Vacuolation in Animals Treated with Polyethylene-Glycol-Conjugated Proteins. Toxicol. Sei. 1998, 42, 152-157). The tendency of polyethylene to crystallize can therefore lead to deposits in the kidney. The elimination through the kidneys also limits the molecular weight of PEG for medical therapeutics to the renal cutoff size, which for globular proteins is approximately 50 to 60 kDa.

The present inventors have reported in 2014 on the copolymerization of ethylene oxide with glycidyl methyl ether (GME) by use of the monomer activation method and tetraoctylammonium bromide and tris-iso-butylaluminum as initiators (see Frey et al, “A Challenging Comonomer Pair: Copolymerization of Ethylene Oxide and Glycidyl Methyl Ether to Thermoresponsive Polyethers”, Macromolecules 2014, 47, 5492-5500). Rather broad molecular weight distributions were obtained (dispersity in the range of 1.21 to 1.5), which are clearly not suitable for medical applications. As discussed at the time, with the conventional anionic ring-opening method only molecular weights up to 3000 g/mol and high dispersities have been reported for the polymerization of GME. In previous studies regarding the homopolymerization of GME to poly(glycidyl methyl ether) (PGME) it was generally stated that polymerization causes difficulties, and no molecular weight control can be achieved by anionic ring-opening polymerization as employed for medical PEG (Frey et al., Biomacromolecules 2014, 15, 1935-1954). In all available reports regarding GME polymerization to date, either phosphazene bases were used as a catalyst or aluminum compounds had to be added in the way of a “monomer activated polymerization”. The same applies to the reaction as published by Labbe et al. (Macromol. Symp. 2007, 249-250, 392-397) using monomer-activated ring-opening polymerization for synthesis of polyglycidylether. This technique suffers from a common side reaction described in FIG. 11. The undesired transfer of hydride leads to further initiating species, therefore limiting the required high end-group fidelity. Additionally, the occurrence of initiation by iso-propyl via monomer-activated ring-opening polymerization is also reported in the literature e.g. in Billouard, C.; Carlotti, S.; Desbois, P.; Deffieux, A. “Controlled” High-Speed Anionic Polymerization of Propylene Oxide Initiated by Alkali Metal Alkoxide/Trialkylaluminum Systems. Macromolecules 2004, 37 (11), 4038-4043. DOI: 10.1021/ma035768t and Herzberger, J.; Niederer, K.; Pohlit, H.; Seiwert, J.; Worm, M.; Wurm, F. R.; Frey, H. Polymerization of Ethylene Oxide, Propylene Oxide, and Other Alkylene Oxides: Synthesis, Novel Polymer Architectures, and Bioconjugation. Chemical reviews 2016, 116 (4), 2170-2243. DOI: 10.1021/acs.chemrev.5b00441. None of these polymers or copolymers are suitable for medical applications for several reasons: (i) traces of phosphazene bases lead to toxicity and cannot be removed; (ii) dispersities exceed 1.1, which is prohibitive for most medical applications and (iii) end-group fidelity of the polymers derived from the methods is insufficient for attachment to peptides, drugs or lipids via bioconjugation. Therefore, neither PGME homopolymer nor copolymers of GME have been considered for PEGylation type applications to date.

The formation and retention of well-defined polymer end-groups (end-group fidelity) is a critical tool for chain extension and all post polymerization reactions. End group fidelity is also a decisive factor for the use of polymers in bioconjugates for pharmaceutical applications, in which the biomolecules are connected by means of the end-groups. Polymers with low end-group fidelity comprise a large amount of macromolecules that are not bound to the biomolecule and cannot be removed from the mixture without undue expenditure. The formed conjugates do not have the required purity. The loss of end-groups is therefore not desirable but cannot be completely avoided. End-group fidelity varies greatly depending on the reaction conditions in the preparation of the polymers.

Polyether copolymers having a main chain derived from ethylene oxide and an oligooxyethylene side chain are known in the art. They are usually used in applications for solid electrolytes, for example in batteries. Most of the polymers described have high or very high molecular weights and a high dispersity. U.S. Pat. No. 6,162,563 A may be mentioned as example, which discloses such polymers. In the working examples polymers with molecular weights above 1,000,000 g/mol are prepared. The dispersities are not disclosed, but a heterogenous catalyst comprising tributyl tin chloride and tributyl phosphate is used. The polymers therefore have high dispersities.

Isono Takuya et al, “Design and synthesis of thermoresponsive aliphatic polyethers with a tunable phase transition temperature”, Polymer Chemistry, Vol. 8, no. 37, 1 Jan. 2017, pages 5698-5707 discloses design and synthesis of thermoresponsive poly(glycidyl ether) homopolymers and copolymers with varying side chain structure, molecular weight and main chain tacticity and their thermoresponsive properties. U.S. Pat. No. 6,162,563 A discloses solids electrolytes comprising copolymers of ethylene oxide and glycidyl ethers of —(CH₂—CH₂—O)ₙ—R¹ with R¹=alkyl with 1 to 12 carbon atoms.

### Purpose of the Invention

It is the objective of the present invention to provide alternative polymers that are able to replace PEG in its applications and that can alleviate at least some of the aforementioned disadvantages of PEG. The alternative polymer should especially be suited to replace PEG in pharmaceutical applications. They should have low tendency to crystallize. It is further an objective to provide polymers with low dispersity. A further objective is to provide polymers with high end-group fidelity. It is also desired to provide a process that allows preparation of such polymers in a simple and commercially feasible way. The alternative polymers should have a low affinity towards anti-PEG antibodies (APAs). A further goal is to provide polymers with a low immunogenicity.

### DETAILED DESCRIPTION

The present invention concerns polyether polymers comprising monomer units that have a structure of formula (I):

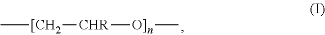

wherein residues R are selected independently from each other from the group consisting of hydrogen, R¹ and R²;
wherein residues R¹ are selected independently of each other from the group consisting of

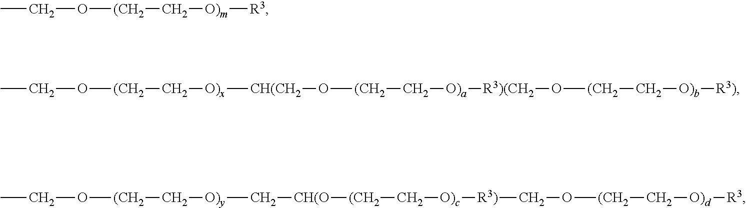

wherein m are independently of each other 1, 2, 3, 4 or 5 and a, b, c, d, x and y, are independently from each other 0 or 1;
wherein R² is —CH₂—O—R³,
wherein residues R³ are selected independently of each other from the group consisting of hydrogen, methyl, ethyl, n-propyl and iso-propyl;
wherein at least 3% of the residues R are hydrogen
and wherein at least 3% of the residues R are residues R¹, based on the total number of all residues R in the polymer;
wherein the polyether polymers are statistical copolymers and wherein at least 97% of the monomer units of the polyether polymers are of formula (I), based on all monomer units of the polyether polymers;
   characterized in that
      the polyether polymers have at least a number average molecular weight in the range of 1,000 to 50,000 g/mol as determined by MALDI-TOF MS and the dispersity of the polyether polymers is 1.15 or less as measured by size exclusion chromatography in DMF by calibration with PEG standards. n may be any integer, as long as the molecular weight requirements are met. It is preferably an integer in the range of 10 to 1000. These new polyether polymers are a new type of material, which behaves very different to PEG in regard to the affinity towards anti-PEG antibodies (APAs) and have a low immunogenicity. They also have a low crystallinity. But the polyether polymers of the present invention behave very similar to PEG with respect to various features that are important for pharmaceutical applications, including aqueous solubility, hydration, cell viability and biocompatibility. In the present patent application polymers of the present invention are also addressed as “scPEG” (poly(ethylene oxide) derivatives with side chains) and the corresponding monomers with side chains are also addressed as “scEO”.

Key to the reduced interaction with APAs is the use of substituents on the PEG backbone, i.e. side chains. The increased spatial requirements of the said PEG copolymers impede or disable interaction with anti-PEG antibodies according to the specific “lock and key principle”. Besides the steric impact, the random or statistical distribution of the substituents over the polymer backbone and/or the random or statistical steric orientation of the substituents furthermore impede the development and formation of specific anti-polymer antibodies.

Key to the similarity of the features of PEG and the inventive polymers is the use of alkoxymethyl side chains, which are structurally close to PEG. This maintains water solubility and other properties in view of PEG. A repeating group of the present polymer wherein R comprises an oligooxyethylene side chain with a methoxy end group, e.g. —CH₂—O—(CH₂—CH₂—O)ₘ—CH₃ has the same molecular formula as the same amount of repeating units of PEG (—CH₂—CH₂—O)ₘ₊₁— and is therefore a constitutional isomer of m+1 main chain repeating units of PEG. It comprises the same number of atoms and very similar functional groups. This leads to similar properties, especially to similar water solubility, which is important for applications in biological systems. The steric hindrance provides low crystallinity. The same is true for all other side chains R of the polymers of the present invention including the side chains with a glycerol ether partial structure.

Methoxymethyl, Ethoxymethyl, n-propoxymethyl and iso-propoxymethyl may be used as residue R or R², respectively, to adjust the properties of the polymers further, for example their solubility, interaction with lipids, properties in respect to the passage of biological membranes etc. However, Ethoxymethyl, n-propoxymethyl and iso-propoxymethyl lead to a reduced solubility in water. The choice of such substituents also leads to a deviation from the behavior of PEG for other properties. For this reason, the amount of repeating units with the residues selected from the group consisting of ethoxymethyl, n-propoxymethyl and iso-propoxymethyl is preferably limited to 50% of all residues R of the polymer of formula (I), preferably to 30%, more preferably to 10% and most preferably to 1%. Also in embodiments of the present invention in which at least 10% of residue R of the polymers are selected from the group consisting of ethoxymethyl, n-propoxymethyl and iso-propoxymethyl at least 10% are hydrogen or R¹ and more preferred at least 25% are hydrogen or R¹. In a further preferred embodiment only up to 20% of the residues R may be selected from the group consisting of ethoxymethyl, n-propoxymethyl and iso-propoxymethyl. These residues are not required for some of the uses of the polymers of the present invention in pharmaceutical applications. For this reason, polymers of the present invention are preferred where R is selected from the group consisting of hydrogen, R¹ and methoxymethyl. Obviously, ethylene oxide repeating units in the inventive polymers also lead to similar properties with PEG. This selection therefore provides the polymers that are closest in behavior to PEG.

Higher homologues of methoxymethyl, i.e. where R² is C4 alkoxymethyl and higher lead to increasingly low solubility in water and other detrimental properties and should therefore generally be avoided. They may however be used in small quantities when required, preferably in amounts of 5% or less and more preferably in amounts of 3% or less and most preferred 1% or less, based on the sum of all residues R. If the polyether of the present invention comprises higher homologues of methoxymethyl, where R is C4-alkoxymethyl or higher, the same proviso for the presence of hydrogen applies with the same preferred amounts. Various other comonomers may be also used in small amounts, i.e. preferably less than 3 wt %, more preferably less than 1 wt % and most preferred in less than 0.1 wt % based on the number average molecular weight of the polymer of formula [I], if necessary. They may for example be used to introduce functional groups or side chains into the polymer. Best suited for the use in pharmaceutical application are however usually polymers of the present invention that comprise only the repeating units of formula (I).

As mentioned before, ethylene oxide repeating units and glycidyl ether repeating units with ether side chains, provide polymers with very similar properties to poly(ethylene oxide). The properties of the polymers of the present invention are still closer to the properties of PEG, when at least some of the residues R are selected to be hydrogen. Furthermore, immunogenicity of copolymers will be reduced as compared to homopolymers, because they do not only differ in the orientation of side groups, but also in the incorporation of side groups along the polymer backbone. The polymers of the present invention are therefore copolymers.

Hydroxyl groups in the side chains may be used to further modify the polymer or may increase interaction with biological systems. Where this is preferred, hydroxyl groups may be part of the polymers, as defined above. However, if it is desired to reduce interactions with biological systems, the amount of hydroxyl groups should be limited. Preferred are polymers of the present invention where no more than 10%, more preferably no more than 1%, even more preferably 0.1% and most preferred none of the rests R comprise hydroxyl groups, i.e. residues R³ are selected independently of each other from the group consisting of methyl, ethyl, n-propyl and iso-propyl. Furthermore, the aforementioned considerations for the selection of the residue R² apply mutatis mutandis to the residue R³. Therefore, the amount of repeating units in which R³ is selected from the group consisting of ethyl, n-propyl and iso-propyl is preferably limited to 50% of all residues R of the polymer of formula (I), preferably to 30%, more preferably to 10% and most preferably to 1%. Also in embodiments of the present invention in which at least 10% of residue R³ of the polymers are selected from the group consisting of ethyl, n-propyl and iso-propyl preferably at least 10% are hydrogen and more preferred at least 25% are hydrogen. In a further preferred embodiment only up to 20% of the residues R³ may be selected from the group consisting of ethyl, n-propyl and iso-propyl. Most preferred are polymers of the present invention where all residues R³ are methyl. These polymers provide the polymers that are closest in behavior to PEG.

The largest differences between the side groups and therefore the lowest immunogenicity is provided by polymers in which some of the repeating units have no side groups, i.e. where R is hydrogen. In the polyether polymers of the present invention at least 3% of the residues R are hydrogen. More preferred at least 10% of residues R are hydrogen, even more preferred at least 30% of the residues R are hydrogen and most preferred, at least 50% of the residues R are hydrogen.

For some application polyether polymers are preferred in which some of the rests R are R². This may for example be used to adjust the hydrophilicity, crystallinity or other features of the polyether polymers of the present invention. In such a case polyether polymers of the present invention wherein at least 3% of residues R are residues R² may be used. Preferably the amount of residue R² is in the range of 3 to 50%, more preferably 5 to 20% and even more preferably 5 to 10%.

As discussed above, the properties of the polymers of the present invention are most pronounced, if the differences between the monomer units of the backbone are greatest. A combination of monomers where R is hydrogen and R¹ is therefore preferred. Preferably 3 to 95% of the residues R are hydrogen and 3 to 95% of the residues R are R¹. More preferred are 10 to 90% of the residues R are hydrogen and 10 to 90% of the residues R are R¹. Even more preferred are 20 to 80% of the residues R are hydrogen and 20 to 80% of the residues R are R¹. The lowest immunogenicity are providing polymers of the present invention, where about 50% of all residues R are hydrogen. For this reason polymers are preferred where 30 to 70% of the residues R are hydrogen and 30 to 70% of the residues R are R1. Especially useful in pharmaceutical applications is a polyether polymer of the present invention, wherein the residue R is selected from the group consisting of hydrogen and R¹. The aforementioned preferred ranges apply accordingly also to embodiments where R is selected from the group consisting of hydrogen and R¹.

As mentioned above, the present polymers may comprise other monomer units than those of formula (I). However, for pharmaceutical applications as far as possible, a homogeneous composition may be preferred. It is therefore preferred that the polyether polymers of the present invention comprise at least 80% of monomer units that have the structure of formula (I). More preferred are at least 90%, even more preferred at least 98% and most preferred consist the polyether polymers of the present invention of monomer units of formula (I).

In one embodiment, the polyether polymers of the present invention comprise at least 90% of monomer units that have the structure of formula (I) and 10 to 90% of the residues R are hydrogen and 10 to 90% of the residues R are R¹.

In one embodiment, the polyether polymers of the present invention comprise at least 95% of monomer units that have the structure of formula (I) and 50 to 90% of the residues R are hydrogen and 10 to 50% of the residues R are R¹.

In one embodiment, all monomer units of the polyether polymers of the present invention have the structure of formula (I) and 60 to 82% of the residues R are hydrogen and 18 to 40% of the residues R are R¹.

In one embodiment, the polyether polymers of the present invention comprise at least 90% of monomer units that have the structure of formula (I) and 10 to 90% of the residues R are hydrogen and 10 to 90% of the residues R are R¹ and the polyether polymers of the present invention comprises less than 20% of rests R that are R².

In one embodiment, the polyether polymers of the present invention comprise at least 95% of monomer units that have the structure of formula (I) and 50 to 90% of the residues R are hydrogen and 10 to 50% of the residues R are R¹ and the polyether polymers of the present invention comprises less than 10% of rests R that are R².

In one embodiment, the polyether polymers of the present invention comprise at least 95% of monomer units that have the structure of formula (I), the residues R are selected from hydrogen and R¹ and 50 to 90% of the residues R are hydrogen and 10 to 50% of the residues R are R¹.

In one embodiment, all monomer units of the polyether polymers of the present invention have the structure of formula (I), the residues R are selected from hydrogen and R¹ and 60 to 82% of the residues R are hydrogen and 18 to 40% of the residues R are R¹.

A further aspect of the polymers of the present invention is that crystallinity, i.e. degree of crystallization, in comparison to PEG is reduced while retaining the same properties. Some of the polymers have amorphous structure depending on the specific composition. Statistical copolymers of EO with GME or other glycidyl ethers can systematically reduce or completely disable crystallization of PEG. The degree of crystallization of PEG and the related melting point (Table 5) plays a key role in numerous pharmaceutical applications, such as e.g., suppositories or solid dispersions of drugs. In other applications, crystallization is undesired, and fully amorphous PEG is required, for instance in highly hydrophilic and biocompatible polyurethane soft foams used among other purposes for wound dressing. Due to the random incorporation of side chains, the copolymers of this invention permit to obtain fully amorphous PEG copolymer structures. This feature is relevant for biomaterials based on triblock and multiblock structures, for instance combining the polyether with biodegradable polylactide blocks.

Due to their high spatial requirement, the rests R¹ provide have a strong effect on crystallinity and polymers with high content of residues R¹ provide amorphous polymers that are otherwise similar to polyethylene glycol. Preferred are therefore polyether polymers of the present invention, wherein 10 to 50% of residues R are residues R¹. Even more preferred are polyether polymers of the present invention, wherein 18 to 40% of residues R are residues R¹. Especially preferred are polyether polymers of the present invention, wherein 10 to 50% of the residues R are R¹ and the remaining residues R are hydrogen and most preferred are polyether polymer of the present invention, wherein 18 to 40% of the residues R are R¹ and the remaining residues R are hydrogen. These copolymers combine most of the aforementioned advantageous properties. They have low immunogenicity, good water solubility, and low crystallinity and are otherwise very similar to PEG.

n is in the range of 10 to 1000; n is preferably in the range of 20 to 900 and more preferably in the range of 30 to 800. Most preferably n is in the range of 30 to 700. These ranges correspond to the polymer weights of PEG that are most suited for medical applications and that are used in existing medical application for PEG. Furthermore, the polymers of the present invention have preferably a molecular weight Mₙ as determined by MALDI-TOF MS in the range of 1,000 to 50,000 g/mol, more preferably in the range of 2,000 to 50,000 g/mol, even more preferably in the range of 2,000 to 30,000 g/mol and most preferably in the range of 2,000 to 9,000 g/mol. These ranges apply to all embodiments of the present invention.

The polyether copolymers of the present invention may be random copolymers or statistical copolymers. Such copolymers provide the lowest immunogenicity, as they do not provide a blueprint for the immune system for antibodies. They are intrinsically resistant to an immune response and are therefore preferred embodiments of the present invention. These polymers may be prepared by a process of the present invention as discussed below.

As described above, similarity to PEG is an important feature of the polymers of the present invention. This is especially true for the end groups of the polymer. Processing of PEG for the use in the pharmaceutical field, involves regularly the modification of the end groups. For this purpose, any end group known to be used for PEG can also be used for the present polymers. This includes not only the use of all known end groups for PEG, but also their modification including the kind of modification and the process of modification. If necessary, in order to allow the present polymers to function in this regard as similar or in fact identical to PEG, the present polymers may be provided with ethylene oxide repeating units on either end or both ends of the polymer, as depicted in Formula (Ia) to (Ic). These are preferred embodiments of the present invention.

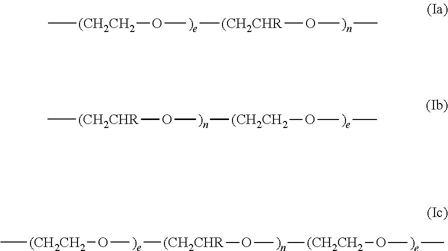

In these formulae the partial formula —(CH₂CHR—O—)ₙ— depicts a polymer of the present invention as described herein, n is preferably 10 to 900 and e may be any number, but is preferable a number in the range of 1 to 10 and more preferably in the range of 1 to 3. In any case the indices n and e must be chosen such that the molecular weight requirements as stated herein are met, i.e. the number average molecular weight of Polymers (Ia) to (Ic) must be in the range of 1,000 to 50,000 g/mol as determined by MALDI-TOF MS. Polymers according to formulae (Ia) to (Ic) allow the modification of the termini of the present polymers exactly as PEG is modified in known processes and especially in present industrial processes. This allows for a smooth transition of established processes from PEG to the polymers of the present invention. Such can be prepared without much additional effort, as will be discussed below. The preferred molecular weight of the polyether copolymers of formulae (Ia), (Ib) and (Ic) and of the polyether copolymers of formulae (II), (IIa), (IIb) and (IIc) is the same as for polyether copolymers of formula (I), including the preferred ranges.

A further embodiment of the present invention, are polyether polymers represented by any of the following formulae (II) and (IIa) to (IIc):

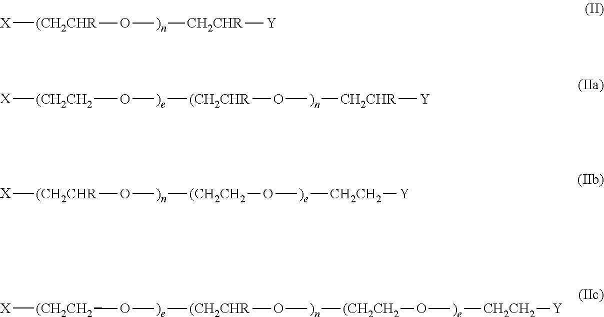

wherein n is 19 to 999 and e is as defined in any of formulae (Ia) to (Ic) and both values of n and e must be chosen with the proviso that the molecular weight requirements are met. R is as defined in formula (I). X is the end group derived from the initiator of the reaction and the α end group (alpha end group) and Y is the ω end group (omega end group).

X or Y or both may be chosen from any end group known for use in PEG. The same end groups may also be provided for the polymers of formulae (Ia) to (Ic).

X and Y may independently of each other or both comprise one or more functional group selected from the group consisting of acetal (dialkoxy), aldehyde (formyl), amide (carboxamido), azide, carbonate (alkoxycarbonyl)oxy), carboxyl (carboxy), carboxylic anhydride, ester (alkoxycarbonyl), ether, halo, haloformyl (carbonohaloridoyl), hemiacetal (alkoxyol), hemiketal (alkoxyol), hydroxy, imide (imido), imine (imino), ketal (dialkoxy), ketone (oyl), orthoester (trialkoxy), primary, secondary, tertiary amino group, primary, secondary and tertiary alkoxy group, sulfhydryl (sulfanyl, H—S—), thioether and combinations thereof. In a preferred embodiment X or Y may be selected from the group consisting of alkyl, hydrogen, hydroxy, alkoxy, sulfanyl, phthalimide, amide, amine and combinations thereof. X or Y or both may be a primary alkoxy group selected of the formula R—CHₙ—O—, wherein R is C1-C25-linear, branched or cyclic alkyl or phenyl and n equals 1 to 20. X and Y may also be a straight or branched C4-C25-alkoxy group, wherein at least one —CH₂— group is exchanged by an oxygen atom, with the proviso that all oxygen atoms are separated from each other by at least two carbon atoms; the C4-C25-alkoxy group is preferably selected from the group consisting of —O—(CH₂—CH₂—O)ₘ—R³, —O—(CH₂—CH₂—O)ₓ—CH(CH₂—O—(CH₂—CH₂—O)ₐ—R³)(CH₂—O—(CH₂—CH₂—O)_{b}—R³) and —O—(CH₂—CH₂—O)_{y}—CH₂—CH(O—(CH₂—CH₂—O)_{c}—R³)—CH₂—O—(CH₂—CH₂—O)_{d}—R³, wherein R³ and a, b, c, d, x and y are as defined above. Preferred are X or Y or both selected from the group consisting of methoxy, ethoxy, propoxy, butoxy, pentoxy, hexoxy, heptoxy, octoxy, nonoxy, decanoxy, 3-ethyl-butoxy, (1,3-dialkoxypropan-2-yl)oxy, 2,3-dialkoxypropoxy and combinations thereof. Especially preferred are MeOCH₂CH₂O—, MeO(CH₂CH₂O)₂—, BenzylOCH₂CH₂O—, BZO(CH₂CH₂O)₂—, (Bz)₂N—CH₂CH₂O—, (Bz)₂N—(CH₂CH₂O)₂—, phthalimide-CH₂CH₂O—, phthalimide-(CH₂CH₂O)₂—, (1,3-dimethoxypropan-2-yl)oxy, (2,3-dimethoxypropan-1-yl)oxy, 2-((1,3-dimethoxypropan-2-yl)oxy)ethan-1-oxy, (2,5,8-trioxanonan-3-yl)methyloxy and (2,6,9-trioxadecan-4-yl)methyloxy. (2,5,8,11-tetraoxadodecan-6-yl)methyloxy and (2,5,9,12-tetraoxatridecan-7-yl)oxy, wherein Me is methyl and Bz is benzyl. X or Y or both may also be the alkoxy residue of an alcohol of the following formula:

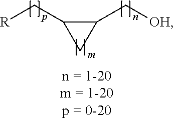

wherein R is hydrogen or linear, branched or cyclic alkyl or phenyl. X or Y or both may be the alkoxy residue of a secondary alcohol selected from any of the formulae

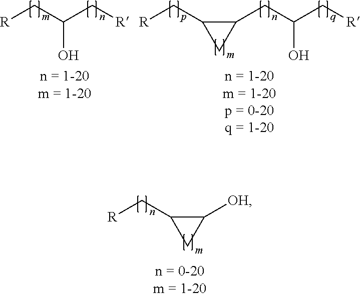

wherein R and R′ are independently from each other hydrogen or linear, branched or cyclic alkyl or phenyl. Among them a secondary alkoxy group is preferred that is selected from the group consisting of 2-propoxy, 2-butoxy, 2-pentoxy, 3-pentoxy, 2-hexoxy, 3-hexoxy, 2-heptoxy, 3-heptoxy, 4-heptoxy and cyclohexyloxy.

X or Y or both may also be selected from primary alkenyloxy groups, which may be linear, branched or cyclic. X or Y or both may also be the alkoxy residue of an alcohol of the following formulae:

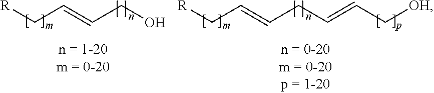

wherein R is hydrogen or linear, branched or cyclic alkyl or phenyl. Among them a primary alkenyloxy group is preferred that is selected from the group consisting of 2-propenoxy (allyloxy), 3-butenoxy, 2-butenoxy, 3-methyl-2-butenoxy, 4-pentenoxy, 3-pentenoxy, 2-pentenoxy and 3,5-hexadienoxy.

X or Y or both may further be selected from secondary alkenyloxy groups, which may be branched or cyclic. X or Y or both may be the alkoxy residue of any of the following formulae:

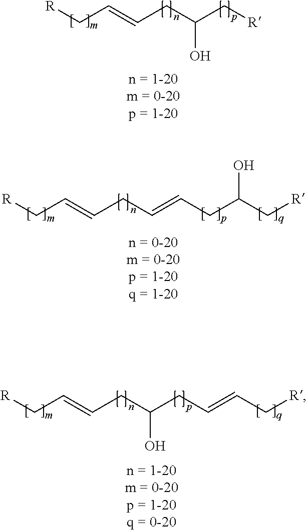

wherein R and R′ are independently from each other hydrogen or linear, branched or cyclic alkyl or phenyl. The secondary alkenyloxy group may be of any of the formulae 4-penten-2-oxy, 3-penten-2-oxy, 5-hexen-2-oxy, 4-hexen-2-oxy, 3-hexen-2-oxy, 4,6-heptadien-2-oxy, 1,4-pentadien-3-oxy.

X or Y or both may further be selected from aryloxy and heteroaryloxy groups. Benzyloxy groups are especially preferred. X or Y or both may be the aryloxy or benzyloxy residue of an alcohol of any of the following formulae:

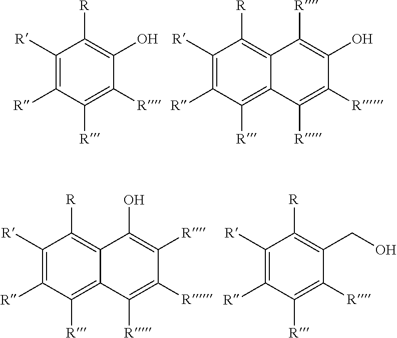

wherein R, R″, R′″, R″″, R′″″ and R″″″ are independently from each other hydrogen or linear, branched or cyclic alkyl or phenyl. X or Y or both are preferably selected from the group consisting of benzyloxy, phenyloxy and naphthyloxy.

X or Y or both may be multifunctional alkoxy groups, i.e. may have multiple ether bonds to the α- or ω-position of a plurality of macromolecules of the polymers of the present invention according to formulae (II) or (IIa) to (IIc). Any suitable polyol may be selected therefore. X or Y or both may especially be selected from the group consisting of polyethers of carbohydrates, especially of Ribose, polyethers of 1,1,1-trimethylolpropane, polyethers of glycerol, polyethers of saccharides or hydrogenated saccharides.

Further, Y may be selected from the group consisting of acrylamide, acrylates, aldehyde, alkyne, amine, aminooxy, azide, benzotriazole carbonate, carboxylic acid, chloroformate, cyanuric chloride, dithioester, epoxide, fluorescein, hydrazide, imidazoyl formate, iminoester, isocyanate, maleimide, mesylate, methacrylate, NHS ester, nitrobenzoate, nitrophenylcarbonate, succinimidyl active ester, succinimidyl carbonate, N-succinimidyl carbonate, succinimidyl succinate, thiocarbonate, thiol, thiol carboxylic acid, tosylate, triflate, vinyl sulfone and xanthate.

It is especially preferred that X is selected from the group consisting of hydrogen, alkyl, hydroxy, sulfanyl, C1-C25-alkoxy, C1-C25-thioalkoxy, amino, amino-C1-C25-alkoxy, dibenzylamino-C1-C25-alkoxy, amide, N-heterocyclic carbenes and N-heterocyclic olefins. It is also especially preferred that Y is selected from the group consisting of hydrogen, hydroxy, alkoxy, —CH₂—C(═O)—R⁴ wherein R⁴ is selected from the group consisting of hydrogen, C1-C20-alkyl and C1-C20-alkoxyl, —CHO, alkylcarbonyloxy, alkyoxycarbonyloxy, amine, C1-C25-monoalkylamine, C1-C25-dialkylamine, carboxamido, azide, halogen, sulfanyl, thioalkoxy and sulfonate. Equally preferred X and/or Y be a straight or branched C4-C25-alkoxy group, wherein at least one —CH₂— group is exchanged by an oxygen atom, with the proviso that all oxygen atoms are separated from each other by at least two carbon atoms, as described herein. In addition, for any alkoxy rest defined herein for the residue X the corresponding thioalkoxy residue may be used, i.e. where a sulfur atom is present instead of the oxygen atom.

Most preferred are polymers of the present invention, where X and/or Y are selected from the group consisting of low molecular weight drugs, nanocarriers, liposomal structures, peptides, polypeptides, glycoproteins, polynucleotide, polysaccharides, lipid structures, liposomes, surfaces and interfaces, which are bonded directly by a covalent bond or by a spacer to the polymer. Nanocarriers usable in the present invention are for example selected from the group consisting of carbon nanotubes (CNT), dendrimers, gold nanorods, lipid nanoparticels, liposomes, micelles, nanocrystals, niosomes, polymeric nanoparticles (PNP), solid lipid nanoparticles (SLNs) and virus-based nanoparticles (VNP).

The polymers of the present invention have dispersities of 1.15 or less. The dispersities of the present invention were determined using size exclusion chromatography (SEC) in DMF by calibration with PEG standards as described in more detail in the examples. They are suitable for applications where low dispersities are required, as for example in pharmaceutical, medical and biological applications. Dispersity of polymers in pharmaceutical, medical and biological applications is of great importance, as macromolecules of different molecular weight may behave differently in biological systems. They may for example differ in solubility in water, membrane permeability, crystallinity and renal clearance. Since a uniform behavior of all molecules is desired in medical applications, a low dispersity is often required. Preferred are therefore polymers of the present invention that have a dispersity of 1.15 or less. Most preferred are polymers of the present invention that have a dispersity of 1.10 or less. This applies to all embodiments of the present invention. A dispersity of 1.10 or less is required for most medical and pharmaceutical applications. Most preferred are polymers of the present invention which have a dispersity that is lower than 1.08.

Preferred embodiments of the present invention are polyether polymers represented by the following formula (II):

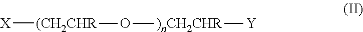

wherein residues R are as defined herein; X is selected from the group consisting of hydrogen, alkyl, hydroxy, sulfanyl, C1-C25-alkoxy, C1-C25-thioalkoxy, amino, amino-C1-C25-alkoxy, dibenzylamino-C1-C25-alkoxy, amide, N-heterocyclic carbenes and N-heterocyclic olefins; Y is selected from the group consisting of hydrogen, hydroxy, alkoxy, —CH₂—C(═O)—R wherein R is as defined above, —CHO, alkylcarbonyloxy, alkyoxycarbonyloxy, amine, alkylamine, carboxamido, azide, halogen, sulfanyl, thioalkoxy, N-succinimidyl carbonate and sulfonate; X and/or Y may also be selected from the group consisting of low molecular weight drugs, nanocarriers, liposomal structures, peptides, polypeptides, glycoproteins, polynucleotides, polysaccharides, lipid structures, liposomes, surfaces and interfaces, which are bonded directly by a covalent bond or by a spacer to the polymer, n is in the range of from 19 to 999, characterized in that the dispersity is 1.15 or less. Especially preferred is N-succinimidyl carbonate. X and Y may also be a straight or branched C4-C25-alkoxy group, wherein at least one —CH₂— group is exchanged by an oxygen atom, with the proviso that all oxygen atoms are separated from each other by at least two carbon atoms, as described above.

An especially preferred embodiment of the present invention is a polyether polymer of the present invention, wherein X is alkoxy and Y is hydroxy. Even more preferably X is a straight or branched C4-C25-alkoxy group, wherein at least one —CH₂— group is exchanged by an oxygen atom, with the proviso that all oxygen atoms are separated from each other by at least two carbon atoms, as described above and Y is hydroxy. Further preferably X is a C1-C10-alkoxy group and Y is hydroxy and even more preferably X is a C1-C3 linear alkoxy group and Y is hydroxy. These embodiments are analogous to commercially available PEG derivates. Most preferred X is methoxy and Y is OH, which is analogous to mPEG. The polyether polymers of the present invention may also have a structure where X and Y are both hydroxy.

As discussed in regard to the dispersity, a unified behavior of all molecules in drugs and excipients used in pharmaceutical and medical applications is preferred. The end-group fidelity of the polymers of the present invention may be determined by MALDI TOF MS or by a combination of MALDI TOF MS with ¹H NMR spectroscopy by known methods. The polymers of the present invention have preferably an end-group fidelity of at least 95% in regard to the group X and more preferably of at least 98%. In a further embodiment the polymers of the present invention have preferably an end-group fidelity of at least 95% in regard to the group Y and more preferably of at least 98%. Even more preferred are polyether polymers of the present invention, wherein the end-group fidelity of the polymer in regard to group X and group Y is at least 95% and most preferably of at least 98%. Especially preferred are polyether polymers of the present invention, wherein the end-group fidelity of the polymer in regard to group X and group Y is at least 95% and the dispersity is 1.10 or less and most preferred wherein the end-group fidelity of the polymer in regard to group X and group Y is at least 98% and the dispersity is 1.10 or less.

In another embodiment of the present invention the polyether polymers are polyether polymers represented by the following formula (II):

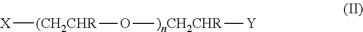

wherein R has the same meaning as described herein, wherein X is selected from group X as mentioned in the other embodiments of (I) and (II) or from the group consisting of hydrogen or X′,
wherein X′ is selected from the group consisting of

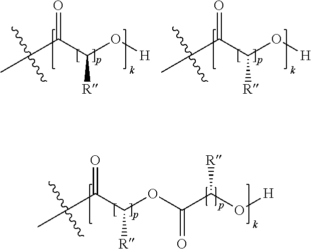

wherein R″=H or alkyl, alkenyl, aryl, alkoxymethyl, alkenyloxymethyl, aryloxymethyl, aminomethyl and thioalkoxymethyl, preferably

### R″ is

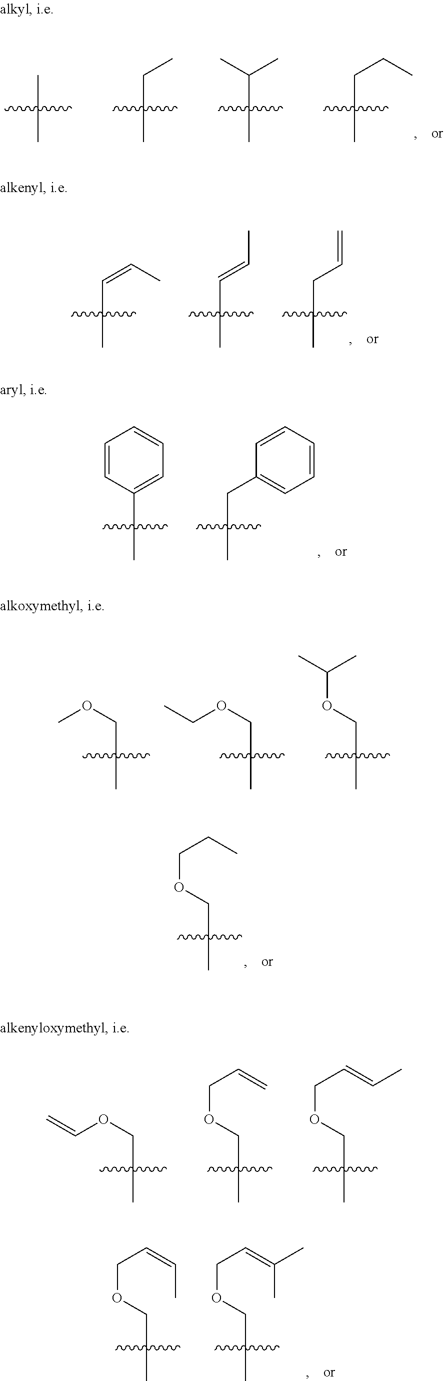

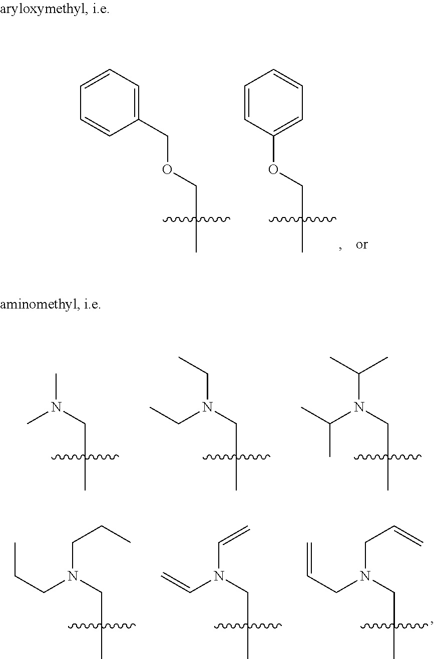

wherein
p=1, 2, 3, 4 or 5, and
k=5-500, preferably 5-250, most preferably 10-200; and

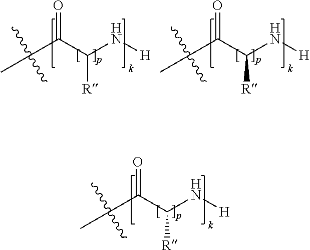

wherein R″=H or alkyl, alkenyl, aryl, alkoxymethyl, alkenyloxymethyl, aryloxymethyl, aminomethyl and thioalkoxymethyl, preferably

### R″ is

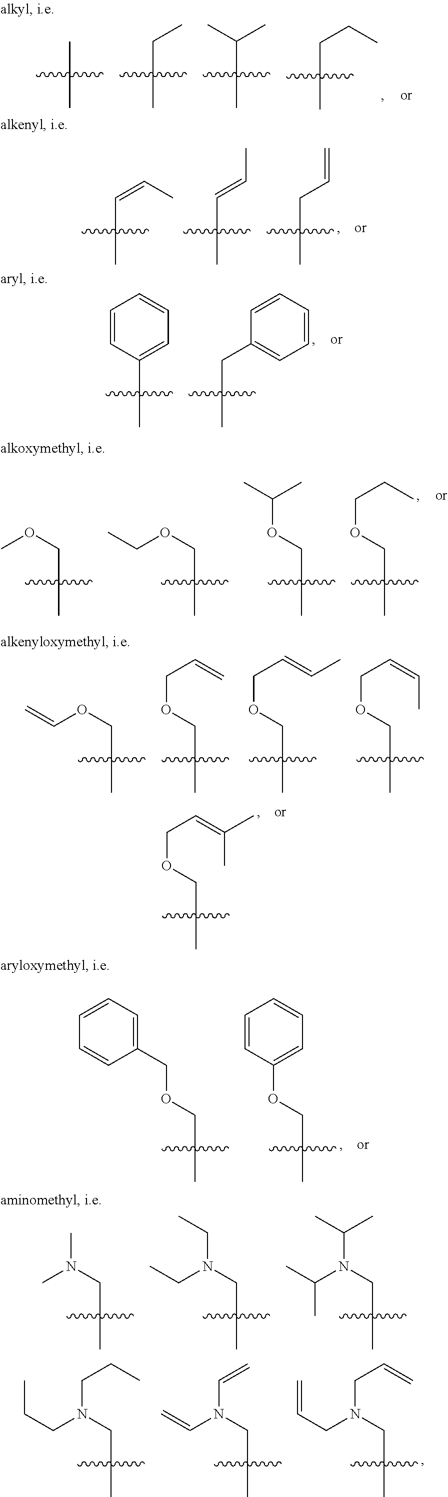

wherein
p=1, 2, 3, 4 or 5 and
k=5-500; preferably 5-250, most preferably 10-200; and

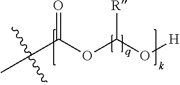

wherein R″=H or alkyl, alkenyl, aryl, alkoxymethyl, alkenyloxymethyl, aryloxymethyl, aminomethyl and thioalkoxymethyl, preferably

### R″ is

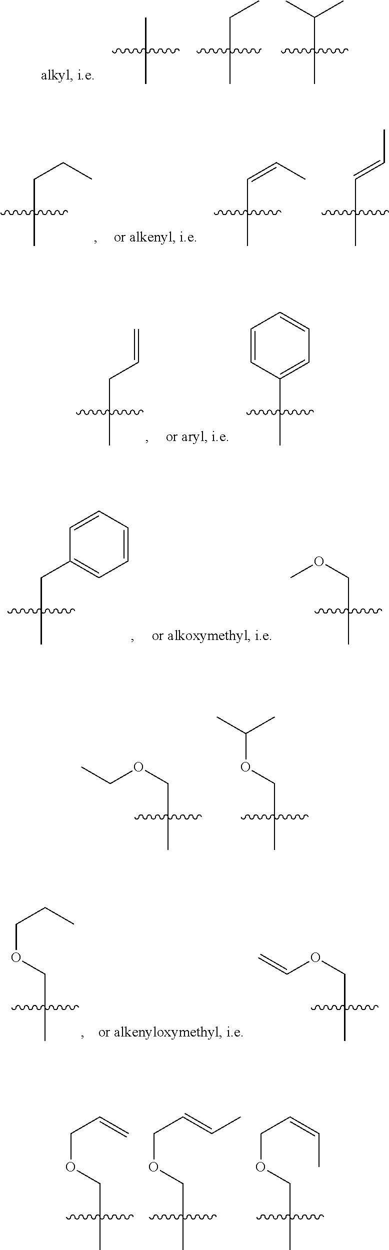

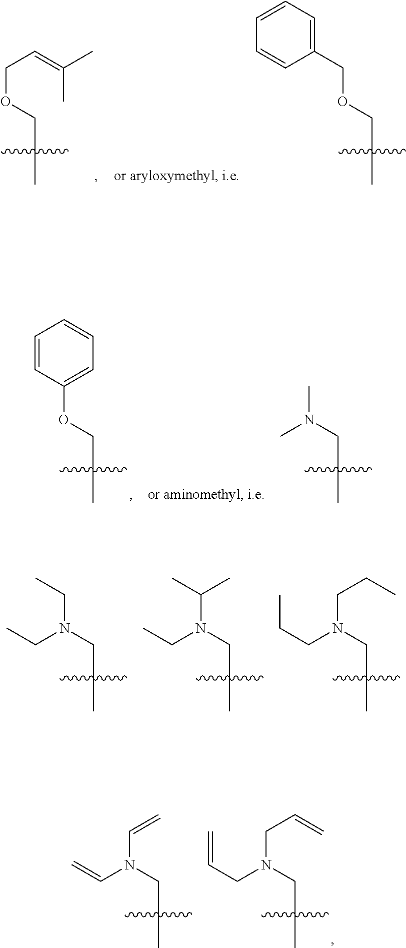

wherein
q=3, 4 or 5 and
k=5-500, preferably 5-250, most preferably 10-200;
and

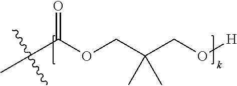

wherein k=5-500, preferably 5-250, most preferably 10-200; and
wherein Y is Z—W meaning composed of substituent Z and W linked via a covalent bond,
wherein

### Z is —O—R′—O— and

W is —R—CHCH₂—(O—CHRCH₂—)ₙ—V;
wherein V is X or X′;
wherein R′ is selected from the group consisting of

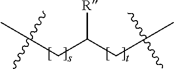

wherein
R″=H or alkyl, alkenyl, aryl, alkoxymethyl, alkenyloxymethyl, aryloxymethyl, aminomethyl and thioalkoxymethyl, preferably

### R″ is

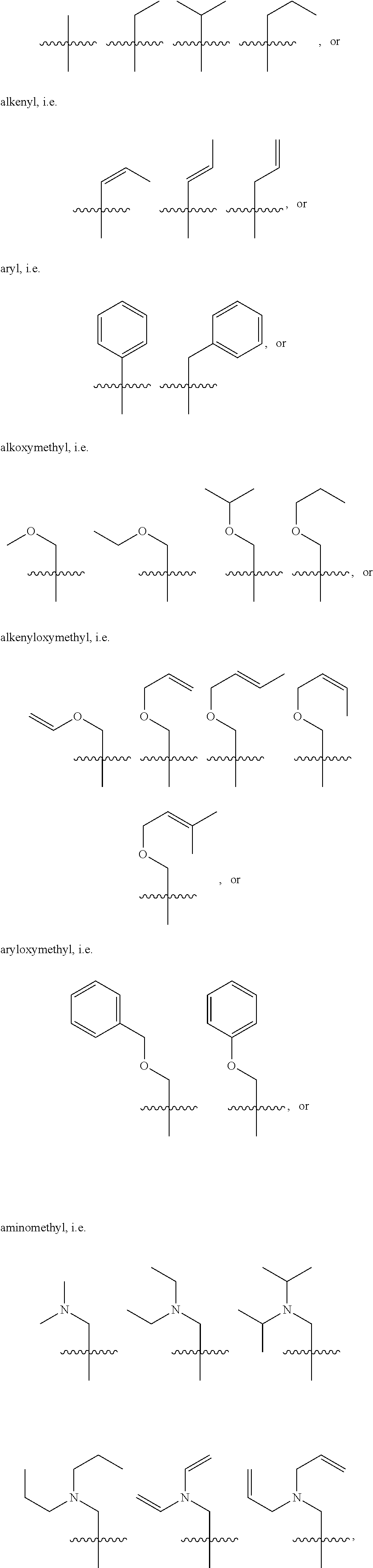

wherein
s=0-20
t=0-20, and
further R′ is

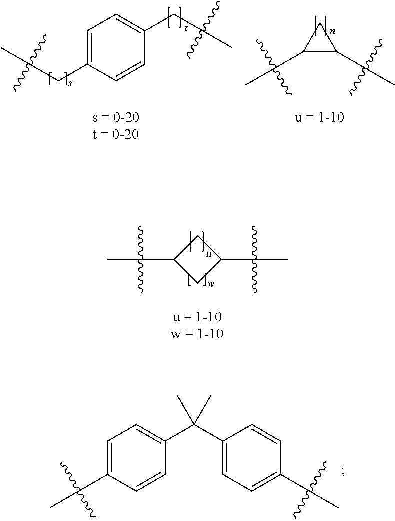

In another embodiment block copolymers of polyether polymers of the present invention and poly(L-lactic acid) (PLLA) are preferred having —O—CH3, —O-Bz, or —OH as functional group at the end of the polymer chain, i.e. diblock polymers MeO-A-b-PLLA-OH, BzO-A-b-PLLA-OH or HO-A-b-PLLA-OH and HO-A-b-PLLA-OH and triblock polymers such as HO-PLLA-b-A-b-PLLA-OH, wherein A is a polyether polymers of the present invention.

In all embodiments of the invention n is preferably in the range of from 10 to 100 for formula (I) and 9 to 999 for formulae (Ia), (Ib), (Ic), (II), (IIa), (IIb) and (IIc), more preferably in the range of 30 to 800, as long as the aforementioned requirements for the molecular weight of the inventive polymers are met. Most preferably n is in the range of 30 to 700 for all polymers of formulae (I), (Ia), (Ib), (Ic), (II), (IIa), (IIb) and (IIc). These ranges correspond to the polymer weights of PEG that are most suited for medical applications and that are used in existing medical application for PEG. The molecular weight is as stated above, including the preferred ranges.

The dispersity of all compounds of the present invention is 1.15 or less measured by size exclusion chromatography in DMF by calibration with PEG standards and preferably 1.10 or less. Preferably the end group fidelity of the polymer in regard to group X and group Y is at least 95% and the dispersity is 1.10 or less and most preferred wherein the end-group fidelity of the polymer in regard to group X and group Y is at least 98% and the dispersity is 1.10 or less.

As aforementioned, the polymers of the present invention have similar properties as PEG, especially in biological systems. The present polymers may therefore substitute PEG in most of his applications. The polymers of the present invention are especially designed for use in the pharmaceutical and medical field. They may also be used with the same benefits in veterinary medicine and in other biological applications. In these fields the polymers of the present invention can fulfill the same functions as PEG but have reduced immunogenicity and antigenicity. Because of their irregular structure, they do not elicit a strong immune response. The inventive polymers show especially a low response to anti-PEG antibodies, as evidenced by ELISA tests. Conjugates used for masking drugs and other pharmaceutically active compounds usually comprise PEG in addition to a physiologically active compound or an adjuvant. The polymers of the present invention may be used instead of PEG in any such application. At the same time the immune response elicited by the present polymers will be weak or non-existent thereby resolving one of the main problems in the use of PEG.

A further embodiment of the present invention is therefore a conjugate comprising a polyether polymer of any of the preceding claims and a substrate. The conjugates of the present invention may in the following also be named polyether-conjugated substrates or polymer-conjugated substrates. In the conjugates of the present invention a polymer of the present invention is bonded to a substrate directly or indirectly by a covalent bond. The polymer of the present invention may be bonded to the substrate by a spacer, which constitutes an indirect bond. The substrate may be selected from the group consisting of a pharmaceutically active compound, an adjuvant, a surface and interfaces. The pharmaceutically active compound is preferably selected from the group consisting of low molecular weight drugs, peptides, polypeptides, proteins, glycoproteins, polynucleotides and polysaccharides. The adjuvant is preferably a vesicle or carrier, which are preferably used to carry a pharmaceutically active compound. The adjuvant is preferably selected from the group consisting of nanocarriers, liposomal structures, lipid structures and liposomes. Nanocarriers usable in the present invention are for example selected from the group consisting of carbon nanotubes (CNT), dendrimers, gold nanorods, lipid nanoparticles, liposomes, micelles, nanocrystals, niosomes, polymeric nanoparticles (PNP), solid lipid nanoparticles (SLNs) and virus-based nanoparticles (VNP). In a further preferred embodiment the substrate is selected from the group consisting of low molecular weight drugs, nanocarriers, liposomal structures, peptides, polypeptides, proteins, glycoproteins, polynucleotides, polysaccharides, lipid structures, liposomes, surfaces and interfaces. Also in these embodiments the substrate may be bonded directly by a covalent bond or by a spacer to the polymer. Especially preferred are conjugates of the present invention with bovine serum albumin.

In the conjugates of the present invention the polyether polymer comprises monomer units that have a structure of formula (I):

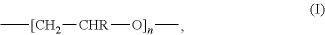

wherein residues R are selected independently from each other from the group consisting of hydrogen, R¹ and R²;
wherein residues R¹ are selected independently of each other from the group consisting of

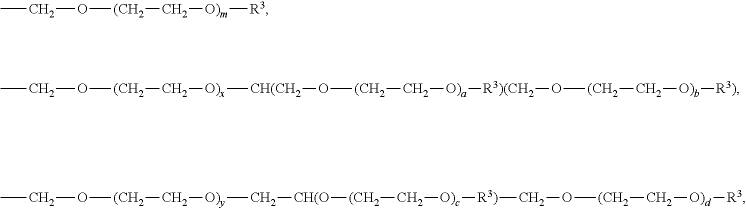

wherein m are independently of each other 1, 2, 3, 4 or 5 and a, b, c, d, x and y, are independently from each other 0 or 1;
wherein R² is —CH₂—O—R³,
wherein residues R³ are selected independently of each other from the group consisting of hydrogen, methyl, ethyl, n-propyl and iso-propyl;
and wherein at least 3% of the residues R are residues R¹, based on the total number of all residues R in the polymer. The dispersity of the polyether polymers is preferably 1.15 or less as measured by size exclusion chromatography in DMF by calibration with PEG standards. The number average molecular weight is preferably in the range of 400 g/mol to 50,000 g/mol. Most preferably the polymers have a dispersity of 1.15 or less and a number average molecular weight in the range of 400 g/mol to 50,000 g/mol. Furthermore, the conjugates of the present invention comprise preferable the polyether polymers of the present invention as described herein, including the preferred embodiments.

The polymer of the present invention may be bonded to the substrate directly by a covalent bond or by a spacer or any kind. Preferably a hydroxy-residue at the ω-terminal of the polymer of the present invention is used to bond a substrate or a spacer to the polymer. This is most economical, as the polymers of the present invention where X is an inert group and Y is OH can be most easily be prepared (see Examples). The conjugation of a substrate to the OH group at the ω-terminal end of PEG is well established. The same processes may be used for the polymers of the present invention. The substrate may however also be bonded to the α-terminal of the polymer. The polymers of the present invention can also be bonded to more than one substrate. This can be done by use of the α-terminal and the ω-terminal of the polymer and by using functional end groups of the polymer or spacer that can bond to a plurality of substrates. This may also be achieved by use of comonomers with functional groups to which a substrate may be bonded. The bond between the polymers of the present invention and the substrate may be prepared by any means currently used for bonding PEG to substrates. It is also possible to bond a plurality of macromolecules of the polymers of the present invention to one substrate. This is especially useful for vesicle of all kinds including liposomes.

Typical examples for PEG conjugates are the lipid nanoparticles that are essential for vaccines against Covid-19 of the firms BioNTech SE and Pfizer Inc. and Moderna. It comprises a PEG conjugate of lipids, and the lipid nanoparticles (LNPs) are used to encapsulate and protect mRNA, which is the pharmaceutically active substance. The PEG in these conjugates may be substituted by the polymers of the present invention, thereby retaining the effects of the PEG polymer in the conjugate and at the same time reducing the immune response to the conjugate. A further vaccine of this kind is the Covid-19 vaccine of Curevac. Further drugs that comprise PEG conjugates wherein the PEG could be substituted by the present polymers are listed in the following.

TABLE 1

| Trade names | Chemical Structure | Indication |
|---|---|---|
| Jivi ® | 60k-PEG recombinant Factor VII | Hemophilia A |
| | antihemophilic factor | |
| Palynziq ® | 2K-PEG-rhu-Phenylalanine | Phenyl-ketonuria |
| | ammonia-lyase, Pegvaliase-pqpz | |
| Adynovate ® | 20K-PEG-Factor VIII | Hemophilia A |
| | Antihemophilic Factor VIII | |
| Revolixys ® kit | 40K-PEG-RNA aptamer + reverse | Anti-coagulation |
| | agent, Factor-IXa blocker, | |
| | Pegnivacogin/Anivamersen | |
| Onicyde ® | 2K-PEG-Liposomal irinotecan | Metastatic pancreatic cancer |
| | hydrochloride trihydrate | |
| Plegridy ® | 20k PEG-Interferon β-1a | Relapsing from multiple |
| | | sclerosis |
| Movantic ® | <1k PEG-Naloxegol | Opioid-induced constipation |
| Omontys ® | 40K-PEG-Erythropoietin-mimetic | Anemia associated with |
| | peptide, Peginesatide | chronic kidney disease |
| Sylatron ® | 12K-PEG-Interferon α-2b | Melanoma |
| Krystexxa ® | 10k-PEG-Uricase, Pegloticase | Gout |
| Cimzia ® | 40k-PEG-Certolizumab | Rheumatoid arthritis, Crohn's |
| | | disease, Axial |
| | | spondyloarthritis and psoriatic |
| | | arthritis |
| Mircera ® | 30K-PEG-erythropoietin (epoetin) | Anemia associated with |
| | β | chronic kidney disease |
| Macugen ® | 40K-PEG-anti-VEGF aptamer, | Age-related macular |
| | Pegaptanib | degeneration |
| Somavert ® | 5K-PEG-rhuGH (human growth | Acromegaly |
| | hormone), Pegvisomant | |
| Neulasta ® | 20K-PEG-Granulocyte colony | Neutropenia |
| | stimulating factor, Pegfilgrastim | |
| Pegasys ® | 40K-PEG-interferon α-2 | Hepatitis C und B |
| PegIntron ® | 12K-PEG-interferon α-2b | Hepatitis C und B |
| Doxil ®/Caelyx ® | 2K-PEG-Liposomal doxorubicin HCl | Cancer |
| Oncaspar ® | 5K-PEGylated L-asparaginase, | Acute lymphoblastic leukemia |
| | Pegaspargase | |
| Adagen ® | 5K-PEG-adenosine deaminase | Severe combined |
| | (bovine), Pegademase | immunodeficiency disease |
| | | (SCID) |
| Irinotecan ® | PEGylated liposomal irinotecan | Metastatic pancreatic cancer |
| | hydrochloride trihydrate | |
| Lonquez ® | 20k PEG-rhG-CSF | Neutropenia |
| Jintrolong ®¹ | Branched 40k PEG hGH | hGH deficiency |
| Neulapeg ® | 20k-PEG rhG-CSF mutein | Neutropenia |
| Rebinyn ®/ | Branched 40k PEG Recombinant | Hemophilia B |
| Refixia ® | factor VIII | |
| Asparlas ® | 5k-PEG L-Asparaginase | Acture lymphoblastic |
| | | leukemia |
| Revcovi ® | 5k-PEG Recombinant B-domain | Hemophilia A |
| | truncated factor VIII | |
| Besremi ®³ | Branched 40k PEG Proline- | Polycythemia vera |
| | interferon α-2b | |
| PEGPH²⁰ ®³ | 30k-PEG recombinant human | Pancreatic cancer |
| | hyaluronidase | |
| Pegargiminase | 20k PEG Arginine deiminase | Hepatocellular carcinoma |
| | Interleukin-2 | |
| NKTR-214 | Branched PEG-20k Fmoc- | Melanoma, rencal cell |
| | succinimidyl carbonate | carcinoma |
| PRX-201³ | Bifunctional PEG2k α- | Fabry disease |
| | Galactosidase A | |
| Peginterferon | 20k PEG recombinant interferon | Hepatitis C and B², Hepatitis |
| lambda-1a | lambda-1a | D³ |
| Abicipar pegol | 20k PEG anti-VEGF DARPin | Neovascular age related |
| | | macular degeneration |
| TransCon GH ®³ | Four-arm-40PEG spacer hGH | Growth hormone deficiency |
| AM0010² | 30k PEG-Interleukin-10 | Metastatic pancreatic cancer |
| Rolontis ®³ | Bifunctional PEG rhG-CSF | Neutropenia |
| Efpeglenatide³ | Bifunctional PEG Exendin-4 | Type-2 diabetes mellitus |
| | (glucagon-like peptide-1 receptor | |
| | agonist) | |
| Pegbelfermin² | 30k PEG fibroblast growth factor- | Fibrosis, liver disorders, |
| | 21 mutein | nonalcoholic steatohepatitis, |
| | | type 2 diabetes mellitus |
| Imrestor ® | 20k PEG recombinant bovine G- | Reducing risk of clinical |
| (veterinary use) | CSF mutein | mastitis |
| Pegfilgrastim | 20K-PEG-Granulocyte colony | Neutropenia |
| biosimilars: | stimulating factor, Pegfilgrastim, | |
| Fulphila (Mylan ®) | rhG-CSF | |
| Lapelga/Pelgraz | | |
| (Apobiologix ®), | | |
| Udenyca | | |
| (Coherus | | |
| Biosciences ®), | | |
| Pelmeg | | |
| (Mundipharma ®), | | |
| Ziextenzo | | |
| (Sandoz ®) | | |
| Esperoct ® | Branched-PEG40k-SA-CMP, | Hemophilia A |
| | enzymatic glycopegylation at O- | |
| | glycan on Ser-750 | |

¹only in China
²Phase II
³Phase III

In another embodiment of the invention the invention relates to a process for the preparation of a polyether polymer by anionic ring-opening copolymerization (AROP) comprising the steps of:
Providing an anion An⁻,
Adding at least one monomer of the formula

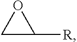

wherein R is as defined for any of formulae (I),
Allowing the polymerization to proceed at a temperature in the range of −10 to 90° C.

In the process of the present invention the anion An⁻ must be an anion suitable for anionic ring-opening copolymerization (AROP). The expert in the art can easily choose among the known anions for this purpose. The An⁻ anion may be selected from the groups consisting of alkyl anion, hydride, OH⁻, alkoxy⁻, SH⁻, thioalkoxy⁻, amine anion, amide anion, imide anion and combinations thereof. Alkyl anions and hydride anions may be provided in the form of metal alkyl or metal hydride compounds. The alkoxy anion and the thioalkoxy anion may be the corresponding anion with the same structure as any of the alkoxy groups and aryloxy groups and aralkoxy groups defined herein for the residue X. Preferably however the alkoxy anion and the thioalkoxy anion are not tertiary alkoxy anions. The imide anion is preferably a phthalimide anion. Polymers of the Formulae (Ia), (Ic), (IIa) and (IIc) may be prepared by first adding the corresponding amount of ethylene oxide to the anion An⁻ and allowing the polymerization to proceed at a temperature in the range of −10 to 90° C. until all of the ethylene oxide is consumed. Subsequently the steps of adding least one monomer of the formula

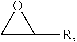

and allowing the polymerization to proceed at a temperature in the range of −10 to 90° C. are performed. In this manner the repeating units adjacent to the α-terminal of the polymers of the present invention are provided with exactly the same structure as PEG. Polymers of the Formulae (Ia), (Ic), (IIa) and (IIc) may also be prepared by use of anions of the formula X—(CH₂CH₂—O—)ₐ⁻, wherein a is as defined for any of the Formulae (Ia), (Ic), (IIa) and (IIc) and X is any residue as defined herein for X. X′ is any residue as defined herein for X′. This is the preferred method. Especially preferred is the use of such anions of the formula X—(CH₂CH₂—O—)ₐ⁻, in which X is alkyl, alkoxy, dialkylamin anion or (RCO)₂N⁻. Even more preferred is the use of anions selected from the group consisting of MeOCH₂CH₂O—, MeO(CH₂CH₂O)₂—, BenzylOCH₂CH₂O—, BzO(CH₂CH₂O)₂—, (Bz)₂N—CH₂CH₂O—, (Bz)₂N—(CH₂CH₂O)₂—, phthalimide-CH₂CH₂O—, phthalimide-(CH₂CH₂O)₂⁻, (1,3-dimethoxypropan-2-yl)oxy anion, (2,3-dimethoxypropan-1-yl)oxy anion, 2-((1,3-dimethoxypropan-2-yl)oxy)ethan-1-oxy anion, (2,5,8-trioxanonan-3-yl)methyloxy and (2,6,9-trioxadecan-4-yl)methyloxy anion. (2,5,8,11-tetraoxadodecan-6-yl)methyloxy anion and (2,5,9,12-tetraoxatridecan-7-yl)oxy anion, wherein Me is methyl and Bz is benzyl. Most preferred are MeO(CH₂CH₂O)₂⁻, BzOCH₂CH₂O⁻ and (Bz)₂N—CH₂CH₂O⁻, (1,3-dimethoxypropan-2-yl)oxy anion, (2,3-dimethoxypropan-1-yl)oxy anion, 2-((1,3-dimethoxypropan-2-yl)oxy)ethan-1-oxy anion, (2,5,8-trioxanonan-3-yl)methyloxy and (2,6,9-trioxadecan-4-yl)methyloxy anion. (2,5,8,11-tetraoxadodecan-6-yl)methyloxy anion and (2,5,9,12-tetraoxatridecan-7-yl)oxy anion.

In addition, in order to synthesize bifunctional initiated copolymers that enable the synthesis of ABA triblock copolymers, a polyether polymer of the present invention bearing two hydroxyl-termini is used as an macroinitiator for ring-opening polymerization of cyclic polyesters, cyclic diesters or cyclic monoesters, e.g. L-lactide, D-lactide, meso-lactide, cyclic carbonates, such as trimethylene carbonate (TMC), 2,2-dimethyl-trimethylene carbonate (DTC), 5-butyl-1,3-dioxan-2-one and 1,3-dioxepan-2-one; glycolide, other cyclic esters and lactams, N-carboxy anhydrides. In an embodiment of the invention the reaction is performed using L-lactide enabling the synthesis of e.g. PLLA-b-P(EG-co-GME)-b-PLLA, wherein PLLA means

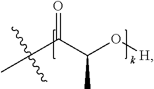

wherein k=5-500, preferably 5-250, most preferably 10-200.

The polymerization is catalyzed by Lewis acids e.g. triethylaluminum (AlEt3) and tin(II) bis-(2-ethylhexanoate) (Sn(Oct)2) or organic bases e.g., DBU (1,8-Diazabicyclo[5.4.0]undec-7-ene) and N-methyl-1,5,7-triazabicyclo[4.4.0]dec-5-ene. The described procedure is not limited to triblock copolymers. With the utilization of a monofunctional P(EG-co-GME) macroinitiator, diblock copolymers (P(EG-co-GME)-b-PLLA) are obtained.

In these embodiments An⁻ can be selected from the group consisting of alkoxy-, thioalkoxy- and amine anion.

Further, in a final step of the preparation, addition of a small amount of pure EO is possible to ensure primary hydroxyl end groups for the ω-terminal of the polymer that can undergo all established coupling strategies to therapeutic entities. This leads to polymers of the present invention with Formulae (Ib), (Ic), (IIb) and (IIc).

The counter ion to the An⁻ anion is preferably selected from the group consisting of Na⁺, K⁺ and Cs⁺. The anion An⁻ may be provided in an inert solvent.

The anionic ring-opening polymerization of epoxides can be conducted in non-protic solvents like DMSO or other polar aprotic solvents, e.g., tetrahydrofuran (THF), methyl-THF, cyclopentyl methyl ether (CPME), benzene, toluene, xylene, HMPA, dioxane, diglyme etc. Surprisingly, it was found that random copolymers can be synthesized by using the non-protic solvent DMSO and statistical copolymers can be synthesized by using polar aprotic solvents e.g. toluene. This can be seen from FIGS. 9, 10, 14 and 15.

The solvent is preferably a non-protic solvent and most preferably dimethyl sulfoxide (DMSO). Also preferably the reaction is performed in the same solvent.

Anionic ring-opening polymerizations with alkyl glycidyl ethers are more prone to undergo chain transfer reactions as follows:

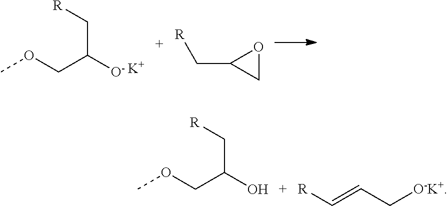

These side reactions increase with the temperature. Therefore, high molecular weight polymers can only be obtained, if the reaction temperature is not above 90° C. The chain transfer reaction results in a broad distribution of molecular weight and therefore in a high dispersity. Therefore, lower polymerization temperatures also provide polymers of the present invention with lower dispersity. On the other hand, alkyl glycidyl ethers are as reactive in the anionic ring-opening polymerization as ethylene oxide. For this reason the polymerization may be performed at temperatures as low as −10° C. Preferably the polymerization is performed at a temperature in the range of 10 to 70° C. and more preferred at a temperature in the range of 10 to 60° C.

A further problem in anionic ring-opening polymerizations is that commercially available alkyl glycidyl ethers are prepared from epichlorohydrin. Part of the epichlorohydrin remains in the alkyl glycidyl ether. Presence of alkyl glycidyl ethers leads to termination reactions as follows:

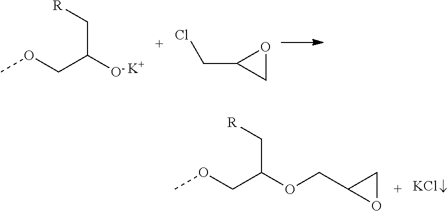

This in turn leads to low molecular weights and high dispersity of the product. Some of the polymers of the present invention, especially those, where R is R² are therefore only accessible by reaction temperatures of −10 to 90° C. or less and by the use of alkyl glycidyl ethers that comprise less than 1 wt % of epichlorohydrin. Preferably the alkyl glycidyl ethers comprise less than 0.5 wt % of epichlorohydrin and more preferably less than 0.1 wt %. Most preferably the alkyl glycidyl ether are free of epichlorohydrin. Removal of epichlorohydrin from the glycidyl methyl ether to the extent necessary is impossible, because the glycidyl methyl ether and epichlorohydrin have similar boiling points, e.g. epichlorohydrin 117.9° C., glycidyl methyl ether 110° C. A procedure for the preparation of epichlorohydrin free alkyl glycidyl ethers is provided in Example 1 of WO 2022/238532 A1. The method may be applied to all monomers used for the preparation of the polyether polymers of the present invention.

Furthermore, high temperatures also lead to side reactions of the starting anion An⁻ or the group X of the living polymer. Limitation of the temperature in the aforementioned range therefore also improves the end-group fidelity at the α-terminal of the polymers of the present invention and allows for end-group fidelities of 95% or more of the X residue. Other known methods for the polymerization of the alkyl glycidyl ethers do not provide the low dispersity required for the present polymers. They also do not allow for the same high end-group fidelity.

Hydroxyl groups in the side chains may be introduced into the polymers of the present invention by use of monomers that have a hydroxyl group that is protected by a protective group, such as for example methoxymethyl ether, tetrahydropyranyl ether, vinyl ether or any other suitable protective group known in the art. After preparation of the polymers, the protective group may be removed to provide a polyether polymer of the present invention with a hydroxyl group in a side chain.

In order to provide random or statistical copolymers or the present invention, it is necessary to add at least two different kinds of monomers to the anion An⁻. The reactivity ratio for both alkyl glycidyl ether employed and ethylene oxide is in the range of 0.7 to 1.3 or 0.6 to 1.7.

A further embodiment of the present invention is the use of the polyether polymers as described for the conjugates of the present invention for the preparation of a conjugate of the polymers with a substrate. The substrate is as defined herein for the conjugates of the present invention. The substrate is preferably a bioactive compound. There is a well-developed chemistry for the preparation of conjugates of PEG, which is known to the expert in the art. The same chemistry may be used for the preparation of a conjugate of the present invention. Preferred is a use of the present invention, for the preparation of conjugated lipids for use in vaccines, in particular based on lipid nanoparticles. These nanoparticles are preferably nanoparticles, as used against COVID-19. Preferred are conjugates of polymers of the present invention with bovine serum albumin (BSA). Especially preferred are conjugates with polymers of the present invention where the y group of the polymer is a N-succinimidyl carbonate group. Even more preferred are conjugates of the present polymer where bovine serum albumin in conjugates by means of such a group Y to a polymer of the present invention.

An additional embodiment for the present invention concerns the preparation of conjugates of the present invention. As discussed above, the conjugates may be prepared by any known process for the preparation of conjugates of PEG. The present invention provides additionally a process, where firstly the process for the preparation of the polymers of the present invention is performed and then a substrate is added to the living polymer, wherein the substrate has a functional group that reacts with the living polymer to form a conjugate.

### Abbreviations

EG ethylene glycol
DCM dichloromethane
DMP2GE 2-(((1,3-dimethoxypropan-2-yl)oxy)methyl)oxirane
DMPEOH 2-((1,3-dimethoxypropan-2-yl)oxy)ethan-1-ol
DMSO dimethyl sulfoxide
EO ethylene oxide
GE glycidyl ether
GME glycidyl methyl ether
scEO ethylene oxide monomers with side chains
scPEG poly(ethylene glycol) polymers with side chains (polymers of the present invention)
SEC size-exclusion chromatography
MALDI ToF MS matrix-assisted laser desorption/ionization time-of-flight mass spectrometry
ELISA enzyme-linked immunosorbent assay

### BRIEF DESCRIPTION OF THE FIGURES

FIG. 1: shows stacked SEC traces of polymers of the present invention synthesized in DMSO.

FIG. 2: shows the SEC trace of a polymer of the present invention synthesized in DMSO.

FIG. 3: shows the MALDI-TOF mass spectrum of a polymer of the present invention synthesized in DMSO.

FIG. 4: shows the ¹H NMR spectrum of a polymer of the present invention synthesized in DMSO.

FIG. 5: shows turbidity measurements of two polymers of the present invention synthesized in DMSO.

FIG. 6: shows stacked ¹H NMR spectra of copolymerization of EO and DMP2GE in DMSO-d₆ at different times of the copolymerization.

FIG. 7: shows the result of a study of the relative reactivities of ethylene oxide and DMP2GE in the anionic ring opening polymerization.

FIG. 8: shows the enzyme-linked immunosorbent assays of mPEG 5k, P(EG-co-GME) and scPEGs.

FIG. 9: shows the SEC trace of a polymer of the present invention synthesized in DMSO.

FIG. 10: shows the MALDI-TOF mass spectrum of a polymer of the present invention synthesized in DMSO.

### EXAMPLES

### Reagents:

Chemicals were purchased from TCI, BLDpharm, Acros Organics, Roth, Sigma Aldrich and Honeywell, unless otherwise noted. Ethylene oxide was obtained from Air Liquide. Glycidyl methyl ether was prepared according to WO 2022/238532 A1, Example 1b. Deuterated solvents were purchased from Deutero GmbH. THF was flashed over basic aluminum oxide before usage. Monomers were either dried over CaH₂ and cryo-transferred or dried over benzene before the polymerizations.

### Measurements:

¹H and ¹³C NMR spectra were recorded on a Bruker Avance III HD 300 or 400 spectrometer with 300 or 400 MHz, respectively, and referenced internally to residual proton signals of the deuterated solvent.

M_{w}, Mₙ and dispersities (M_{w}/Mₙ=PDI) of all samples were determined from the corresponding size exclusion chromatograms (refractive index (RI) detector, DMF, calibration with PEG standards).

Size-exclusion chromatography (SEC) measurements were performed with dimethylformamide (DMF with 1 g/L LiBr) as the mobile phase (flow rate 1 mL/min) on poly(2-hydroxyethylmethacrylat) (PHEMA) 300/100/40 columns at 50° C. Polymer concentrations were 1 mg/mL. Calibration was carried out using poly(ethylene glycol) standards (from Polymer Standard Service, Mainz, Germany).

Differential scanning calorimetry (DSC) measurements were carried out in the temperature range of −90 to 70° C. with a heating rate of 10 K/min at a TA Instruments DSC 250. The thermal history of the samples was excluded via two cooling and two heating cycles. For each sample, the glass transition and the melting temperatures were obtained from the second heating curve.

MALDI-ToF MS measurements were carried out at a Bruker autoflex maX MALDI-TOF/TOF. The potassium salt of trifluoroacetic acid and trans-2-[3-(4-tert-Butylphenyl)-2-methyl-2-propenylidene]malononitrile (DCTB) were utilized as ionization salt and matrix, respectively.

### Example 1: Synthesis of 2-(((1,3-dimethoxypropan-2-yl)oxy)methyl)oxirane (DMP2GE)

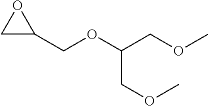

A round bottomed flask equipped with a magnetic stirrer was charged with epichlorohydrin (31.3 g, 26.5 mL, 336 mmol) and cooled with an ice bath. Finely grounded NaOH (20.2 g, 504 mmol) was added under stirring. 1,3-Dimethoxypropan-2-ol (20.2 g, 20.0 mL, 168 mmol) was added and the solution was heated to 30° C. After 5 h, diethyl ether (100 mL) was added. The reaction mixture was filtered and the filtrate was dried over MgSO₄. After an additional filtration step, solvent and excess epichlorohydrin was evaporated under high vacuo. 2-(((1,3-dimethoxypropan-2-yl)oxy)methyl)oxirane (13.4 g, 76.2 mmol, 45%) was obtained after fractional distillation as a colorless liquid.

### Example 2: Synthesis of 2,5,9,12-tetraoxatridecan-7-ol

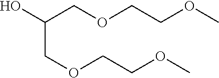

Epichlorohydrin (14.2 g, 12.0 mL, 153 mmol) and 2-methoxy ethanol (46.6 g, 48.0 mL, 612 mmol) were mixed in a three-necked flask equipped with a magnetic stirrer, dropping funnel and reflux condenser. The solution was heated to 50° C. and 50 wt % NaOH solution (8 mL) was added dropwise under stirring. The reaction mixture was heated to 80° C. and stirred overnight. After cooling to room temperature. The reaction mixture was filtered. The filtrate was neutralized with concentrated HCl and extracted with dichloromethane (DCM, 25 mL, five times). The combined organic phases were dried with MgSO₄. After filtration, the solvent was evaporated under vacuo. 2,5,9,12-tetraoxatridecan-7-ol (7.40 g, 35.5 mmol, 23%) was obtained as a colorless liquid after fraction distillation.

### Example 3: Synthesis of 2-(((2,5,9,12-tetraoxatridecan-7-yl)oxy)methyl)oxirane (DMEP2GE)

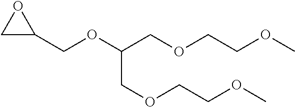 [SMILES: COCCOCC(COCCOC)OCC1CO1]

A round bottomed flask equipped with a magnetic stirrer was charged with epichlorohydrin (6.61 g, 5.60 mL, 71.4 mmol) and cooled with an ice bath. Finely grounded NaOH (4.23 g, 106 mmol) was added under stirring. 2,5,9,12-tetraoxatridecan-7-ol (7.40 g, 35.5 mmol) was added and the solution was heated to 30° C. After 5 h, the solution was cooled to room temperature and diethyl ether (40 mL) was added. The reaction mixture was filtered and the filtrate was dried over MgSO₄. After an additional filtration step, solvent and excess epichlorohydrin was evaporated under high vacuo. 2-(((2,5,9,12-tetraoxatridecan-7-yl)oxy)methyl)oxirane (8.67 g, 32.8 mmol, 92%) was obtained after fractional distillation as a colorless liquid.

### Example 4: Synthesis of 2-((1,3-dimethoxypropan-2-yl)oxy)ethan-1-ol (DMPEOH)

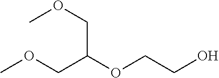

A three necked flask equipped with a reflux condenser and septum was flame dried under high vacuo. NaH (719 mg, 30.0 mmol) was added under argon. The flask was cooled with an ice bath and dry THF (25 mL) was added under argon. The suspension was stirred for 10 min and 1,3-dimethoxypropan-2-ol (3.00 g, 2.97 mL, 25 mmol) was slowly added. After 30 min, 1,3,2-dioxathiolane 2,2-dioxide (3.10 g, 25 mmol) was added in small portions under argon. The resulting reaction mixture was stirred under reflux overnight. After cooling to room temperature THF was evaporated under vacuo. Diethyl ether (25 mL) was added to the residue and diluted H₂SO₄ (50 mL) was added under ice cooling and vigorous stirring. After reaching room temperature overnight, the solution was extracted with diethyl ether (25 mL, three times) and the combined organic phases were dried with MgSO₄. After filtration, the solvent was evaporated. 2-((1,3-dimethoxypropan-2-yl)oxy)ethan-1-ol (711 mg, 4.33 mmol, 17%) was obtained as a colorless liquid after fractional distillation.

### Example 5: General Procedure for the Preparation of Random Copolymers with Branched Side Chains

### Synthesis of α-DMPEO-P(EG_{0.70}-co-DMP2GE_{0.30}) (entry c)

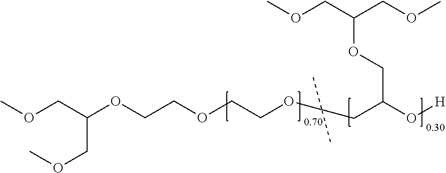

2-((1,3-Dimethoxypropan-2-yl)oxy)ethan-1-ol (59.0 mg, 359 μmol) was dissolved in benzene (5 mL) and transferred via syringe into a flame-dried flask under argon. Potassium-tert-butoxide (39.5 mg, 352 μmol) was dissolved in stabilizer-free THF (3 mL) and small quantities of Millipore water and transferred into the flask. High vacuum was applied to the flask and the solvents were removed under high vacuum. The resulting initiator salt was dried under high vacuum at 55° C. overnight and finally dissolved in dry DMSO (5 mL) under stationary vacuum. After freezing the resulting solution at −78° C., DMP2GE (840 mg, 4.74 mmol) was added via syringe to the flask. EO (488 mg, 503 mL, 11.1 mmol) was added to the flask via cryo-transfer before the cooling bath was removed and the reaction mixture was allowed to warm up to room temperature. The reaction was allowed to stir for 24 h at 30° C. Subsequently, the flask was vented and most DMSO was evaporated under high vacuo. The polymer was redissolved in a toluene and diethyl ether mixture and acetic acid was added. After filtration, solvents and residual acetic acid were evaporated and α-DMPEO-P(EG_{0.70}-co-DMP2GE_{0.30}) (96%) was obtained as a viscous liquid.

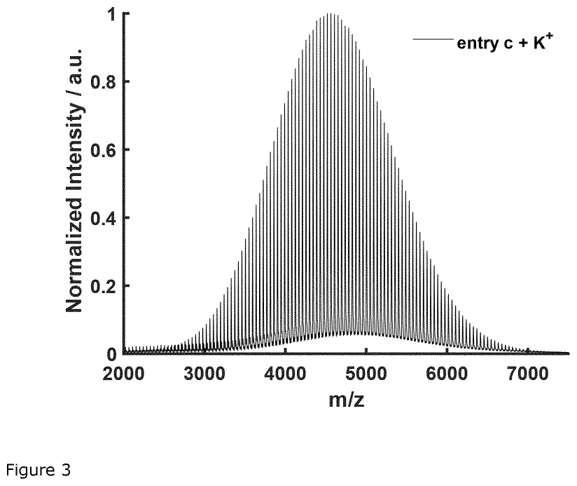

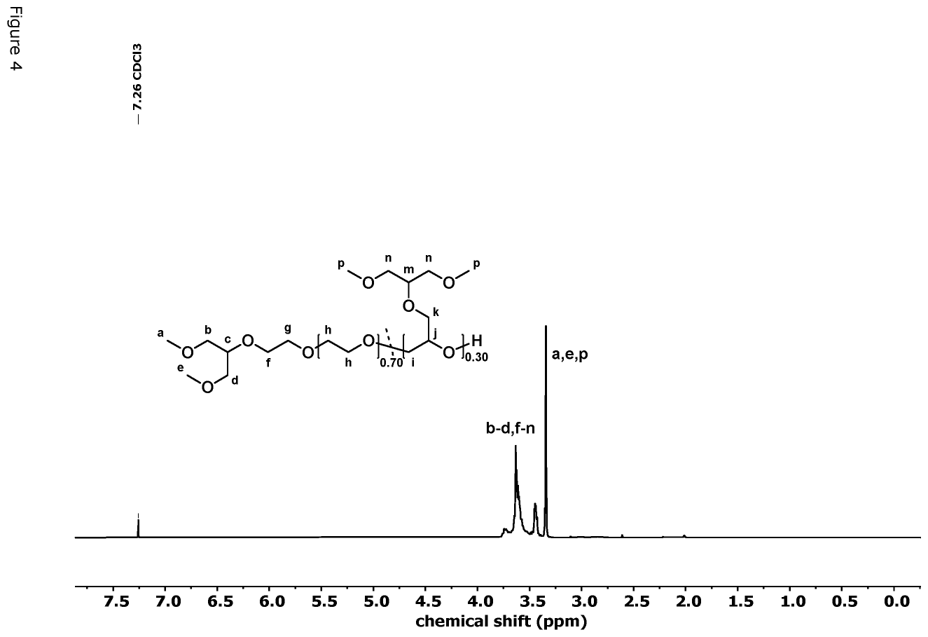

The MALDI-ToF MS of α-DMPEO-P(EG_{0.70}-co-DMP2GE_{0.30}) reveals an exceptional high end-group fidelity (>99%), as can be seen in FIG. 3. The data of the product can be found in Tables 1 and 2, entry a.

Further polymers of the present invention (scPEG) with varying amounts and different glycidyl ether monomers with branched ether side chains (entry a,b and d) were prepared according to the same procedure (entries a, b, and d of Tables 1 to 4).

### Example 6: Competitive Enzyme-Linked Immunosorbent Assay (ELISA)

The interaction of the copolymer samples entry b and c, P(EG-co-GME) and of commercially available mPEG 5k were evaluated by a competitive PEG ELISA kit using a murine monoclonal, horseradish peroxidase conjugated anti PEG antibody (HRP anti-PEG) (Life diagnostics, West Chester, PA, USA). Samples of concentrations ranging from 0 to 6.37 mg ml⁻¹ were prepared in dilution buffer. Additionally, mPEG with a molecular weight of 5000 g mol⁻¹ was utilized as internal standard for comparison. 50 μl of each prepared sample was dispensed to a PEG pre-coated 96-well plate and 50 μl of HRP anti-PEG was added to each well. The solutions were incubated for 1 h at 25° C. with a micro-plate shaker and then washed six times with 300 μl of wash buffer per each well. After removal of residual droplets, 100 μl of 3,3′,5,5′-tetramethylbiphenyl-4,4′-diamine was added to each well and the solutions were mixed on a micro-plate shaker for 20 min. The reaction was stopped by addition of 100 μl of stop solution and the absorbance at 450 nm was read within 5 minutes.

Analysis of ELISA Data: The determined absorbance values were normalized to visualize the percent of maximal binding. The sample concentrations were transformed to a function of log₁₀. The sigmoidal fits were calculated using the following equation with a representing the lower, b the upper limit, x0 the inflection point and dx the hill slope.

y=a+(b−a)/(1+exp((x−x0)/dx))

### Example 7: Kinetics of the Synthesis of P(EG-Co-DMP2GE)

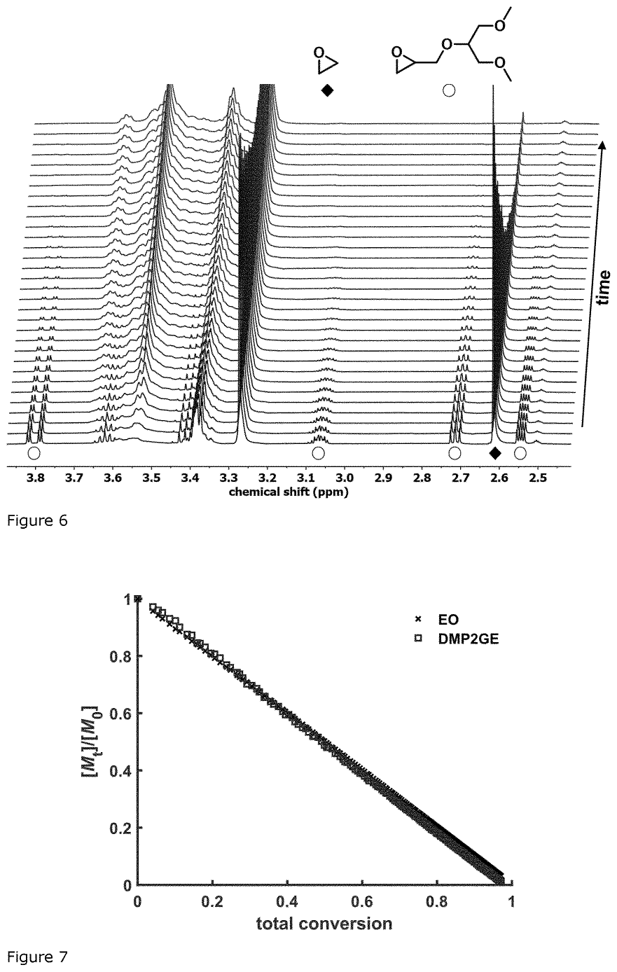

The synthesis of P(EG-co-DMP2GE) was repeated in fully deuterated DMSO for an online in situ ¹H NMR kinetics measurement. DMP2GE and ethylene oxide were dissolved in fully deuterated DMSO. The consumption of ethylene oxide and DMP2GE over time was measured by ¹H-NMR spectroscopy. As measure for the ethylene oxide concentration, the signal of the protons of ethylene oxide at about 2.61 ppm was used. For DMP2GE the signals of the protons at the oxirane ring at about 2.55, 2.71 and 3.08 ppm were used. FIG. 6 shows the decrease of the selected ¹H NMR signals of the two monomers over time. FIG. 7 shows the plot of monomer consumption M_{x,t}/M_{x,t=0} versus total conversion. As can be seen, ethylene oxide and DMP2GE are consumed at nearly the same rate. This data shows that the copolymers of the present invention are almost ideal random copolymers.

### Example 8: Synthesis of 1-methoxy-3-(2-methoxyethoxy)propan-2-ol (MMEPOH)

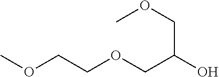

Ethylene glycol monomethyl ether (8.64 g, 9.00 mL, 113 mmol) was added to a flask equipped with a reflux condenser. NaOH solution (19 M, 3 mL) was added under stirring and the resulting solution was heated to 55° C. Glycidyl methyl ether (5.00 g, 5.10 mL, 56.7 mmol) was added and the solution was stirred overnight. The solution was cooled to room temperature and extracted with dichloromethane (DCM, 50 mL) three times. The combined organic phases were dried over magnesium sulphate (MgSO₄). After filtration, the solvent was evaporated under reduced pressure. 1-Methoxy-3-(2-methoxyethoxy)propan-2-ol (3.74 g, 22.7 mmol, 40%) was obtained as a colorless liquid after fractional distillation of the residue.

### Example 9: 1-Chloro-3-(2-methoxyethoxy)propan-2-ol

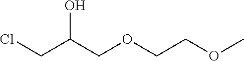

Concentrated sulfuric acid (95%, 2.71 g, 26.3 mmol) was mixed with 2-methoxyethanol (20.0 g, 263 mmol). The mixture was added dropwise to epichlorohydrin (48.6 g, 526 mmol) under stirring while cooling the flask with an ice/water bath. After complete addition of the alcohol, the reaction mixture was stirred at 80° C. while the reaction progress was monitored via TLC. After 16 h, water (30 mL) was added to the reaction mixture. The organic layer was separated and the aqueous phase extracted three times with chloroform (20 mL). The combined organic phases were neutralized with saturated sodium hydrogen carbonate solution, dried over sodium sulfate. Chloroform and epichlorohydrin were removed under reduced pressure. The crude product was distilled under high vacuum. 1-Chloro-3-(2-methoxyethoxy)propan-2-ol was obtained as a colorless liquid (11.34 g, 67.3 mmol, 26%).

### Example 10: 2-((2-Methoxyethoxy)methyl)oxirane (MEGE)

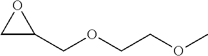

1-Chloro-3-(2-methoxyethoxy)propan-2-ol (11.3 g, 67.3 mmol) was dissolved in diethyl ether (10 mL) and cooled with a water bath. Freshly ground sodium hydroxide (2.96 g, 74.0 mmol) was added to the solution. The reaction progress was monitored via TLC. After completion, the resulted sodium chloride was filtered and the filtrate was dried over sodium sulfate. After removal of diethyl ether under reduced pressure, the crude product was fractionally distilled under high vacuum yielding 2-((2-methoxyethoxy)methyl)oxirane as a colorless liquid (7.62 g, 57.7 mmol, 86%).

### Example 11: General Procedure for the Preparation of Random Copolymers with Linear Side Chains

### Synthesis of α-MMEPO-P(EG_{0.91}-co-MEGE_{0.09}) (entry e)

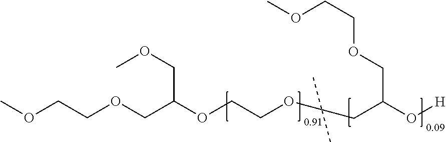

1-Methoxy-3-(2-methoxyethoxy)propan-2-ol (50.0 mg, 300 μmol) was dissolved in benzene (6 mL) and transferred via syringe into a flame-dried flask under argon. Potassium-tert-butoxide (31.0 mg, 270 μmol) was dissolved in stabilizer-free THF (3 mL) and small quantities of Millipore water and transferred into the flask. High vacuum was applied to the flask and the solvents were removed under high vacuum. The resulting initiator salt was dried under high vacuum at 60° C. overnight and finally dissolved in dry DMSO (10 mL) under stationary vacuum. After freezing the resulting solution at −78° C., MEGE (460 mg, 3.47 mmol) was added via syringe to the flask. EO (1.38 g, 1.42 mL, 31.2 mmol) was added to the flask via cryo-transfer before the cooling bath was removed and the reaction mixture was allowed to warm up to room temperature. The reaction was allowed to stir overnight at room temperature. Subsequently, the flask was vented, quenched with acidified MeOH and the solvents were evaporated under high vacuo. The polymer was redissolved in MilliQ water. After lyophilization, α-MMEPO-P(EG_{0.91}-co-MEGE_{0.09}) was obtained as a viscous liquid.

### Discussion of the Results

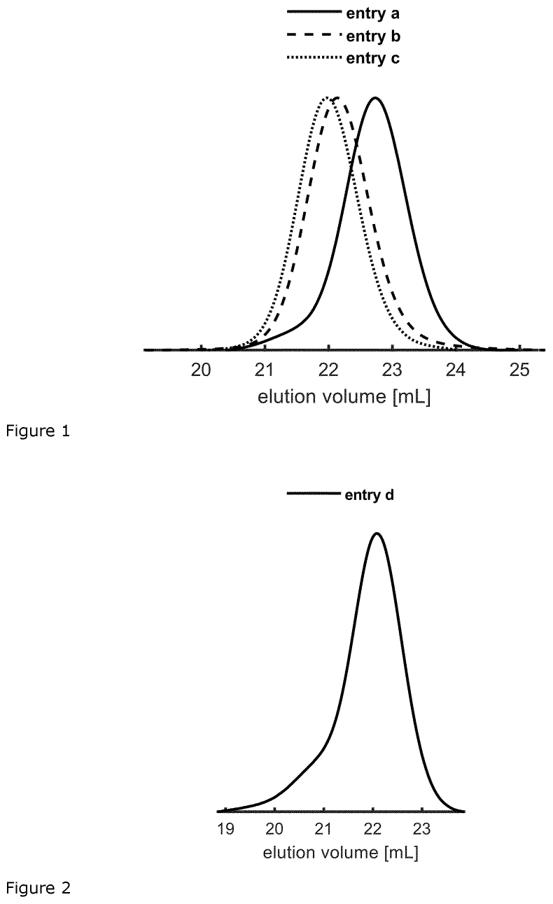

Tables 1, 2, 3 and 4 and FIG. 1 to 5 show the properties of the polymers prepared. They are discussed in the following.

TABLE 1
Composition of polymers prepared

| Entry | | | | | |
|---|---|---|---|---|---|
| (Example) | Sample | DP_{EO NMR} | DP_{GE NMR} | mol %_{EO NMR} | mol %_{scEO NMR} |
| a (5) | α-DMPEO-P(EG_{0.92}- | 46 | 4 | 92 | 8 |
| | co-DMP2GE_{0.08}) | | | | |
| b (5) | α-DMPEO-P(EG_{0.80}- | 43 | 11 | 80 | 20 |
| | co-DMP2GE_{0.20}) | | | | |
| c (5) | α-DMPEO-P(EG_{0.70}- | 38 | 16 | 70 | 30 |
| | co-DMP2GE_{0.30}) | | | | |
| d (5) | α-DMPEO-P(EG_{0.83}- | 43 | 9 | 83 | 17 |
| | co-DMEP2GE_{0.17}) | | | | |
| e (11) | α-MMEPO-P(EG_{0.91}- | 84 | 8 | 91 | 9 |
| | co-MEGE_{0.09}) | | | | |

DP = degree of polymerization.

TABLE 2
Characterization data of polymers prepared

| | | | | | | End- |
|---|---|---|---|---|---|---|
| Entry/ | | M_{n, NMR} | M_{n, MALDI} | M_{n, SEC} | | group |
| (Example) | Sample | [kg/mol] | [kg/mol] | [kg/mol] | PDI_{SEC} | fidelity_{MALDI} |
| a | α-DMPEO-P(EG_{0.92}- | 2.9 | 2.9 | 2.4 | 1.06 | >99% |
| (5) | CO-DMP2GE_{0.08}) | | | | | |
| b | α-DMPEO-P(EG_{0.80}- | 4.0 | 4.0 | 3.0 | 1.06 | >99% |
| (5) | CO-DMP2GE_{0.20}) | | | | | |
| C | α-DMPEO-P(EG_{0.70}- | 4.7 | 4.6 | 3.2 | 1.05 | >99% |
| (5) | CO-DMP2GE_{0.30}) | | | | | |
| d | α-DMPEO-P(EG_{0.83}- | 4.4 | 4.4 | 3.3 | 1.10 | >99% |
| (5) | CO-DMEP2GE_{0.17}) | | | | | |
| e | α-MMEPO-P(EG_{0.88}- | 5.0 | 5.0 | 4.2 | 1.06 | 99% |
| (11) | CO-MEGE_{0.12}) | | | | | |

Mₙ = molecular weight (number average molar mass), PDI = dispersity.

TABLE 3
Thermal properties in aqueous solution

| Entry/ | | [scEO] | Cloud Point |
|---|---|---|---|
| (Example) | Sample | (mol %)^{a} | (°C) |
| a (5) | α-DMPEO-P(EG_{0.92}-co- | 8 | N/O* |
| | DMP2GE_{0.08}) | | |
| b (5) | α-DMPEO-P(EG_{0.80}-co- | 20 | N/O* |
| | DMP2GE_{0.20}) | | |
| c (5) | α-DMPEO-P(EG_{0.70}-co- | 30 | 94 |
| | DMP2GE_{0.30}) | | |
| d (5) | α-DMPEO-P(EG_{0.83}-co- | 17 | N/O* |
| | DMEP2GE_{0.17}) | | |

*N/O = not observed in the range of 0 to 99 °C.

Table 3 shows the thermal properties of the respective compounds in aqueous solution. The polymers were prepared according to Example 5 with varying amounts of the glycidyl ether monomers with ether side chains (scEO). The cloud point of the random copolymers of ethylene oxide and glycidyl ether monomers with ether side chains were investigated via turbidimetry measurements of copolymers containing different amounts of glycidyl ether monomers with ether side chains. Cloud point was defined at a transmittance of 50%.

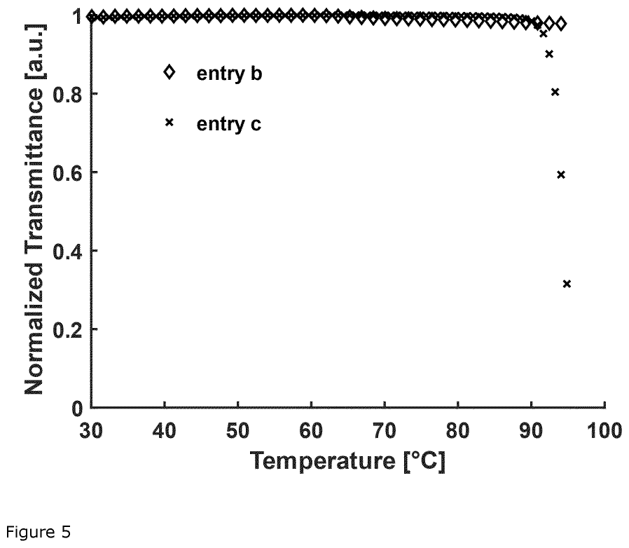

A cloud point of the copolymers was merely observed for 30 mol % DMP2GE, see FIG. 5. Despite the decrease in the cloud point with increasing mol % DMP2GE, solubility in water under physiologically conditions is ensured for all copolymers.

### Dispersity

As can be seen from Table 2, entries a to d, all polymers of the present invention have a very low dispersity, i.e. 1.10 or lower. For visualization, the SEC traces of these polymers are shown in FIGS. 1 and 2. It can clearly be seen that the present invention provides polymers with a very narrow molecular weight distribution.

### Preparation and Purity of the Polymers of the Present Invention

FIG. 4 shows the ¹H NMR spectrum of α-DMPEO-P(EG_{0.70}-co-DMP2GE_{0.30}), i.e., the polymer of Example 5 (entry c of Tables 1 and 2). The product is as obtained from Example 5, without further purification. As can be seen, the present method does not only provide polymers of narrow molecular weight distribution, but also very pure polymers without the need for laborious purification steps. This is an important feature for the use in pharmaceutical applications.

### End-Group Fidelity

As can be seen from Table 2, the end-group fidelity for all polymers of the present invention is nearly 100%. FIG. 3 shows the MALDI TOF mass spectrum of α-DMPEO-P(EG_{0.70}-co-DMP2GE_{0.30}), i.e., the polymer of Example 5 (entry c of Tables 1 and 2) with potassium cation. The only peaks visible here are the peaks of the product molecule. There are no peaks from macromolecules with a different end group. This shows that end-group fidelity of the polymers of the present invention is very high. In addition, the spectrum confirms the narrow molecular weight distribution.

### Solubility

FIG. 5 shows the cloud point measurement of α-DMPEO-P(EG_{0.80}-co-DMP2GE_{0.20}) and α-DMPEO-P(EG_{0.70}-co-DMP2GE_{0.30}), i.e., the polymers of Example 5 (entry b and c of Tables 1 and 2). The cloud point determines the temperature at which immiscibility in water (and therefore in aqueous solution) is observed upon heating. As an example, for the copolymer with 30% DPM2GE (entry c of Tables 1 and 2), the cloud point can be observed at 94° C., showing excellent water solubility of the polymers of the present invention. The homopolymer PEG shows a cloud point of ca. 100° C. For copolymers of <30 mol % glycidyl ether monomers with ether side chains incorporation no cloud point was detected in water at temperatures ranging from 0 to 99° C. Thus, the ether side chains of glycidyl ether monomer repeating units hardly affect water solubility, which is a feature of the similar structure of polyethylene glycol and polymers of the present invention. This is an important feature of the polymers of the present invention as it shows that they can substitute PEG in aqueous systems without causing problems with phase separation or even precipitation.

### Crystallinity

Table 4 shows thermal properties of some of the polymers of Tables 1 and 2:

TABLE 4
Thermal properties in bulk

| Sample | T_{g} [°C] | Tₘ [°C] | ΔH_{PEG} [J/g] | X_{c, PEG} [%] |
|---|---|---|---|---|
| a | −65 | 16 | 48.3 | 24 |
| b | −66 | N/O | N/O | N/O |
| c | −66 | N/O | N/O | N/O |
| d | −67 | N/O | N/O | N/O |

T_{g} = Glass transition temperature; Tₘ = Melting point; ΔH_{PEG} = Enthalpy of fusion; X_{c} = Crystallinity (all measurements by DSC).

As can be seen, crystallinity of the inventive polymers is low and can be completely interrupted by the incorporation of glycidyl ether monomers with ether side chains in the polymer structure. This prevents for example accumulation of the polymers in the kidney or in the liver.

Immunogenicity: Comparison of scPEGs with mPEG and P(EG-co-GME)

TABLE 5
ELISA results

| Entry/ | | Relative APA |
|---|---|---|
| (Example) | Sample | Affinity |
| — | mPEG 5k | 1 |
| — | α-BzO-P(EG_{0.76}-co-GME_{0.24}) | 0.06 |
| b (5) | α-DMPEO-P(EG_{0.80}-co-DMP2GE_{0.20}) | 0.006 |
| c (5) | α-DMPEO-P(EG_{0.70}-co-DMP2GE_{0.30}) | 0.001 |

Relative APA affinities are calculated from the IC₅₀ values of the investigated polymers.

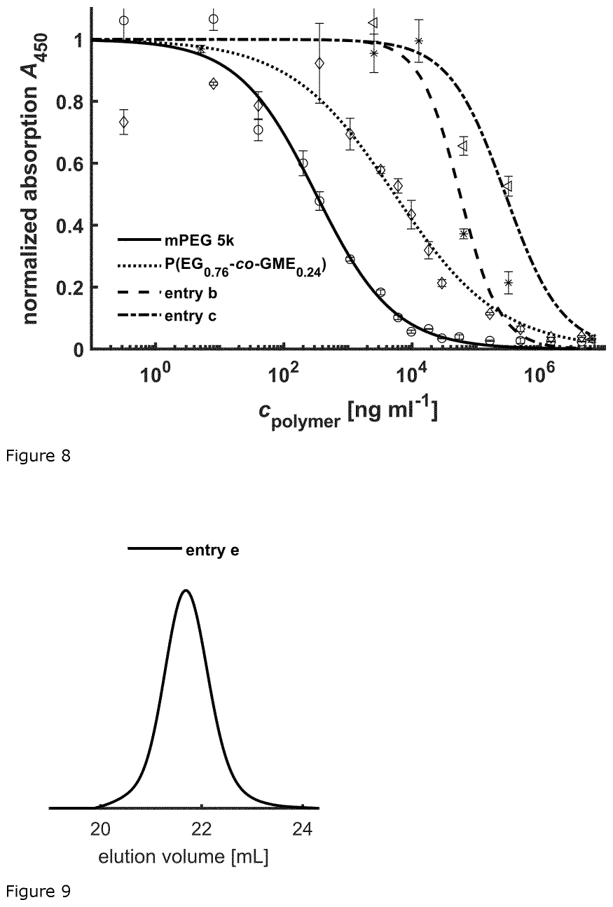

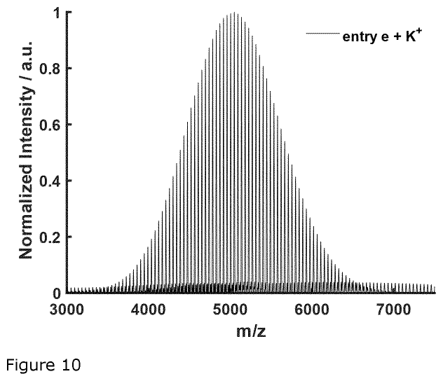

FIG. 8 and Table 5 show the ELISA test results of entry b and c, P(EG-co-GME) and of commercially available mPEG 5k. FIG. 8 shows the function of the normalized absorption at a wavelength of 450 nm versus the log 10 function of the polymer concentration in nanograms per ml, therefore illustrating the anti-PEG antibody interaction with the investigated polymer concentrations. Table 5 illustrates the APA affinity of the investigated scPEGs (entry b and c) and P(EG-co-GME) (prepared according to WO 2022/238532 A1, Example 2, Table 3, Entry d) relative to mPEG 5k. The ELISA data shows a strong influence of the sterically demanding side chains of scPEGs (it is important to note that the x-axis has a logarithmic scale). With increasing amount of scEO incorporated into the polyether structure, significantly higher polymer concentrations are necessary to observe interactions between the copolymer and the APA. Remarkably, a significant stronger hindrance of APA binding was observed for scPEGs in comparison to P(EG-co-GME) with similar amount of incorporated comonomer.

1. Polyether polymers comprising monomer units that have a structure of formula (I): 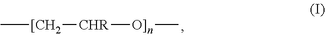
wherein residues R are selected independently from each other from the group consisting of hydrogen, R¹ and R²;
wherein residues R¹ are selected independently of each other from the group consisting of 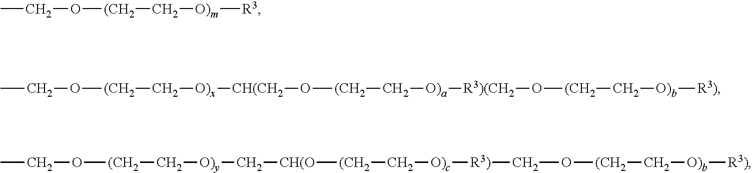
wherein m are independently of each other 1, 2, 3, 4 or 5 and a, b, c, d, x and y are independently from each other 0 or 1;
wherein R² is —CH₂—O—R³,
wherein residues R³ are selected independently of each other from the group consisting of hydrogen, methyl, ethyl, n-propyl and iso-propyl;
wherein at least 3% of the residues R are hydrogen
and wherein at least 3% of the residues R are residues R¹, based on the total number of all residues R in the polymer;
wherein the polyether polymers are statistical copolymers and wherein at least 97% of the monomer units of the polyether polymers are of formula (I), based on all monomer units of the polyether polymers;
wherein
the polyether polymers have at least a number average molecular weight in the range of 1,000 to 50,000 g/mol as determined by MALDI-TOF MS and the dispersity of the polyether polymers is 1.15 or less as measured by size exclusion chromatography in DMF by calibration with PEG standards.

2. Polyether polymers according to claim 1 wherein at least 3% of residues R are residues R².

3. Polyether polymers according to claim 1, wherein 3% to 95% of residues R are hydrogen and 3% to 95% of residues R are residues R¹.

4. Polyether polymers according to claim 1, wherein 10 to 50% of residues R are residues R¹.

5. Polyether polymers according to claim 1 represented by the following formula [II]: 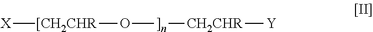
wherein R is as in any claim 1;
X is selected from the group consisting of hydrogen, alkyl, hydroxy, sulfanyl, C1-C25-alkoxy, C1-C25-thioalkoxy, amino, amino-C1-C25-alkoxy, dibenzylamino-C1-C25-alkoxy, amide, N-heterocyclic carbenes, N-heterocyclic olefins;
Y is selected from the group consisting of hydrogen, hydroxy, alkoxy, —CH₂—C(═O)—R⁴ wherein R⁴ is selected from the group consisting of hydrogen, C1-C25-alkyl and C1-C25-alkoxy, —CHO, alkylcarbonyloxy, alkyoxycarbonyloxy, amine, C1-C25-monoalkylamine, C1-C25-dialkylamine, carboxamido, azide, halogen, sulfanyl, thioalkoxy and sulfonate;
X and/or Y may also be a straight or branched C4-C25-alkoxy group, wherein at least one —CH₂— group is exchanged by an oxygen atom, with the proviso that all oxygen atoms are separated from each other by at least two carbon atoms; and
X and/or Y may also be selected from the group consisting of low molecular weight drugs, nanocarriers, liposomal structures, peptides, polypeptides, proteins, glycoproteins, polynucleotide, polysaccharides, lipid structures, liposomes, poly-(lactic acid), polyglycolids, surfaces and interfaces, which are bonded directly by a covalent bond or by a spacer to the polymer.

6. Polyether polymer according to claim 5, wherein X is alkoxy and Y is hydroxy.

7. Polyether polymer according to claim 5, wherein X and Y are each hydroxy.

8. Polyether polymers according to claim 1 represented by the following formula [II]:
X—[CH₂CHR—O]ₙ—CH₂CHR—Y [II]
wherein R is as defined as in claim 1
wherein X is selected from the group consisting of hydroxyl or —O—X′,
wherein X′ is selected from the group consisting of 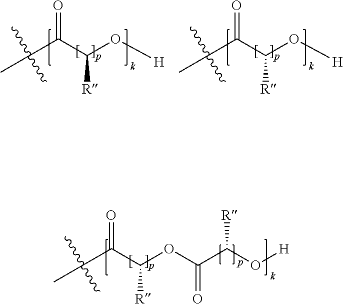
wherein R″=H or alkyl, alkenyl, aryl, alkoxymethyl, alkenyloxymethyl, aryloxymethyl, aminomethyl and thioalkoxymethyl and
p=1, 2, 3, 4 or 5, and
k=5-500; 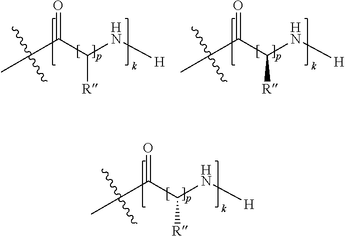
wherein R″=H or alkyl, alkenyl, aryl, alkoxymethyl, alkenyloxymethyl, aryloxymethyl, aminomethyl and thioalkoxymethyl, and
p=1, 2, 3, 4 or 5 and
k=5-500; and 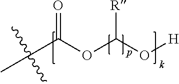
wherein R″=H or alkyl, alkenyl, aryl, alkoxymethyl, alkenyloxymethyl, aryloxymethyl, aminomethyl and thioalkoxymethyl
q=3, 4 or 5 and
k=5-500;
and 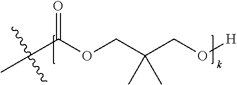
wherein k=5-500; and
wherein Y is —Z—W, wherein
Z is —O—R′—O— and
W is —CH₂CHR—(O—CH₂CHR—)_{n′}—V;
wherein V is X or X′;
wherein R′ is selected from the group consisting of 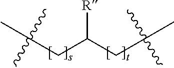
wherein
R″=H or alkyl, alkenyl, aryl, alkoxymethyl, alkenyloxymethyl, aryloxymethyl, aminomethyl and thioalkoxymethyl, and
s=0-20
t=0-20; and
further R′ is selected from

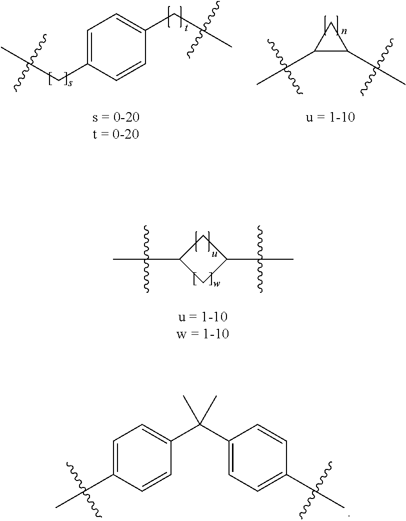

9. Polyether polymer according to claim 5, wherein end-group fidelity of the polymer in regard to group X and/or in regard to group Y is at least 95%.

10. Process for the preparation of polyether polymers according to claim 1 by anionic ring-opening copolymerization comprising the steps of:
providing an anion An⁻,
adding at least one monomer of the formula R, where in R is as defined in claim 1, in the amounts as described in claim 1 and adding optionally further monomers,
allowing the polymerization to proceed at a temperature in the range of −10 to 90° C.

11. Conjugate, comprising a polyether polymer and a substrate, wherein the polyether polymer comprises monomer units that have a structure of formula (I): 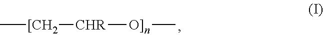
wherein residues R are selected independently from each other from the group consisting of hydrogen, R¹ and R²;
wherein residues R¹ are selected independently of each other from the group consisting of 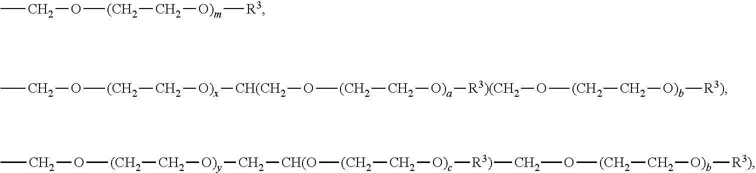
wherein m are independently of each other 1, 2, 3, 4 or 5 and a, b, c, d, x and y, are independently from each other 0 or 1;
wherein R² is —CH₂—O—R³,
wherein residues R³ are selected independently of each other from the group consisting of hydrogen, methyl, ethyl, n-propyl and iso-propyl;
and wherein at least 3% of the residues R are residues R¹, based on the total number of all residues R in the polymer; and the polyether polymer is a polyether polymer according to claim 1 or a polyether polymer prepared by a process comprising the steps of:
providing an anion An⁻,
adding at least one monomer of the formula R, where in R is as defined in claim 1, in the amounts as described in claim 1 and adding optionally further monomers,
allowing the polymerization to proceed at a temperature in the range of −10 to 90° C.

12. Conjugate according to claim 11, wherein the substrate is selected from the groups consisting of low molecular weight drugs, nanocarriers, liposomal structures, peptides, polypeptides, proteins, glycoproteins, polynucleotides, polysaccharides, lipid structures, liposomes, surfaces and interfaces.

13. A method for the preparation of a conjugate, the method comprising conjugating polyether polymers with a bioactive compound, wherein the polyether polymer comprises monomer units that have a structure of formula (I): 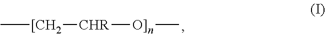
wherein residues R are selected independently from each other from the group consisting of hydrogen, R¹ and R²;
wherein residues R¹ are selected independently of each other from the group consisting of 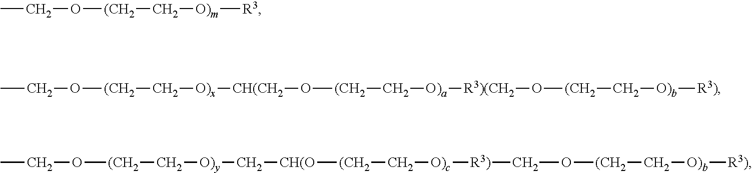
wherein m are independently of each other 1, 2, 3, 4 or 5 and a, b, c, d, x and y, are independently from each other 0 or 1;
wherein R² is —CH₂—O—R³,
wherein residues R³ are selected independently of each other from the group consisting of hydrogen, methyl, ethyl, n-propyl and iso-propyl;
and wherein at least 3% of the residues R are residues R¹, based on the total number of all residues R in the polymer.

14. A method for the preparation of conjugated lipids for use in vaccines, the method comprising conjugating polyether polymers with lipid nanoparticles, wherein the polyether polymers comprise monomer units that have a structure of formula (I): 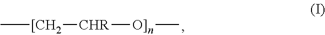
wherein residues R are selected independently from each other from the group consisting of hydrogen, R¹ and R²;
wherein residues R¹ are selected independently of each other from the group consisting of 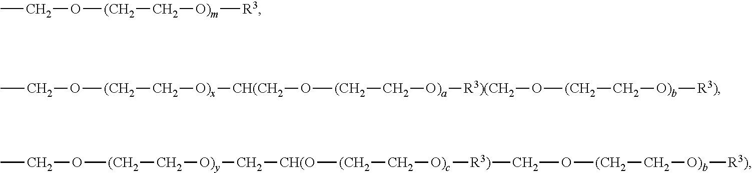
wherein m are independently of each other 1, 2, 3, 4 or 5 and a, b, c, d, x and y, are independently from each other 0 or 1;
wherein R² is —CH₂—O—R³,
wherein residues R³ are selected independently of each other from the group consisting of hydrogen, methyl, ethyl, n-propyl and iso-propyl;
and wherein at least 3% of the residues R are residues R¹, based on the total number of all residues R in the polymer.

15. A method for the preparation of a conjugate, the method comprising conjugating the polyether polymers according to claim 1 with a bioactive compound.

16. A method for the preparation of a conjugate, the method comprising conjugating the polyether polymers prepared by the process according to claim 10 with a bioactive compound.

17. A method for the preparation of conjugated lipids for use in vaccines, the method comprising conjugating the polyether polymers according to claim 1 with lipid nanoparticles.

18. A method for the preparation of conjugated lipids for use in vaccines, the method comprising conjugating the polyether polymers prepared by the process according to claim 10 with lipid nanoparticles.

19. The method according to claim 14, wherein the nanoparticles are nanoparticles used against COVID-19.
